SEQUENCE LISTING (17 sequences)

SEQ ID NO: 1 - protein, 5 aa, Artificial Sequence
  Synthetic Construct
DTYIH

SEQ ID NO: 2 - protein, 17 aa, Artificial Sequence
  Synthetic Construct
RIYPTNGYTRYADSVKG

SEQ ID NO: 3 - protein, 11 aa, Artificial Sequence
  Synthetic Construct
WGGDGFYAMDY

SEQ ID NO: 4 - protein, 11 aa, Artificial Sequence
  Synthetic Construct
RASQDVNTAVA

SEQ ID NO: 5 - protein, 7 aa, Artificial Sequence
  Synthetic Construct
SASFLYS

SEQ ID NO: 6 - protein, 9 aa, Artificial Sequence
  Synthetic Construct
QQHYTTPPT

SEQ ID NO: 7 - protein, 120 aa, Artificial Sequence [sequence omitted]

SEQ ID NO: 8 - protein, 107 aa, Artificial Sequence [sequence omitted]

SEQ ID NO: 9 - protein, 5 aa, Artificial Sequence
  Synthetic Construct
NYYIH

SEQ ID NO: 10 - protein, 17 aa, Artificial Sequence
  Synthetic Construct
WIYPGDGNTKYNEKFKG

SEQ ID NO: 11 - protein, 10 aa, Artificial Sequence
  Synthetic Construct
DSYSNYYFDY

SEQ ID NO: 12 - protein, 17 aa, Artificial Sequence
  Synthetic Construct
KSSQSLLNSRTRKNYLA

SEQ ID NO: 13 - protein, 7 aa, Artificial Sequence
  Synthetic Construct
WASTRES

SEQ ID NO: 14 - protein, 8 aa, Artificial Sequence
  Synthetic Construct
TQSFILRT

SEQ ID NO: 15 - protein, 119 aa, Artificial Sequence
  Synthetic Construct
EVQLVQSGAEVKKPGASVKVSCKASGYTFTNYYIHWVRQAPGQGLEWIGWIYPGDGNTKYNEKFKGRATLTADTSTSTAYLELSSLRSEDTAVYYCARDSYSNYYFDYWGQGTLVTVSS

SEQ ID NO: 16 - protein, 112 aa, Artificial Sequence
  Synthetic Construct
DIVMTQSPDSLAVSLGERATINCKSSQSLLNSRTRKNYLAWYQQKPGQPPKLLIYWASTRESGVPDRFSGSGSGTDFTLTISSLQAEDVAVYYCTQSFILRTFGQGTKVEIK

SEQ ID NO: 17 - protein, 142 aa, Homo sapiens
CHQLCARGHCWGPGPTQCVNCSQFLRGQECVEECRVLQGLPREYVNARHCLPCHPECQPQNGSVTCFGPEADQCVACAHYKDPPFCVARCPSGVKPDLSYMPIWKFPDEEGACQPCPINCTHSCVDLDDKGCPAEQRASPLT

(19) United States
(12) Patent Application Publication
(10) Pub. No.: US 2022/0098325 A1
(43) Pub. Date: Mar. 31, 2022

(54) TREATMENT OF CANCER WITH HER2XCD3 BISPECIFIC ANTIBODIES IN COMBINATION WITH ANTI-HER2 MAB

(71) Applicant: Genentech, Inc., South San Francisco, CA (US)

(72) Inventors: Teemu T. JUNTTILA, San Mateo, CA (US); Stuart LUTZKER, South San Francisco, CA (US)

(21) Appl. No.: 17/473,401

(22) Filed: Sep. 13, 2021

Related U.S. Application Data
(63) Continuation of application No. PCT/US2020/022665, filed on Mar. 13, 2020.
(60) Provisional application No. 62/818,556, filed on Mar. 14, 2019.

(51) Int. Cl. C07K 16/32; A61P 35/00; C07K 16/28; A61K 45/06; A61K 39/395

(52) U.S. Cl. CPC C07K 16/32; A61P 35/00; C07K 16/2809; A61K 2039/507; A61K 39/3955; A61K 39/39558; A61K 45/06

(57) ABSTRACT

The present invention provides methods of treating of HER2-positive cancers (such as HER2-positive breast cancer and HER2-positive gastric cancers) using HER2 antibodies, such as a combination of a HER2 T cell-dependent bispecific antibody (TDB) with an additional HER2 antibody (e.g., trastuzumab).

### SEQUENCE LISTING

The instant application contains a Sequence Listing which has been submitted electronically in ASCII format and is hereby incorporated by reference in its entirety. Said ASCII copy, created on Sep. 9, 2021, is named 50474-197003_Sequence_Listing_09_10_21_ST25 and is 8,439 bytes in size.

### FIELD OF THE INVENTION

The present invention relates to the treatment of HER2-positive cancers using HER2 antibodies, such as a combination of a HER2 T cell-dependent bispecific antibody (HER2 TDB) with another HER2 antibody.

### BACKGROUND

Cancers are characterized by the uncontrolled growth of cell subpopulations. Cancers are the leading cause of death in the developed world and the second leading cause of death in developing countries, with over 14 million new cancer cases diagnosed and over eight million cancer deaths occurring each year. According to the American Cancer Society, an estimated 1,762,450 new cases of cancer and 606,880 deaths from cancer will occur in the United States in 2019. As the elderly population has grown, the incidence of cancer has concurrently risen, as the probability of developing cancer is more than two-fold higher after the age of seventy. Cancer care thus represents a significant and ever-increasing societal burden.

Human epidermal growth factor receptor 2 (HER2)-positive cancers, such as breast cancer and gastric cancer, represent some of the most common cancers in the world. Locally advanced and metastatic HER2-positive breast and gastric cancers largely remain incurable diseases, with most patients progressing after receiving HER2-targeted therapies. While important advances have been achieved with the introduction of new anti-cancer agents, overall survival has been only marginally improved, and the long-term prognosis of patients with HER2-positive cancers who experience disease progression during or following a first-line treatment regimen remains dismal.

Thus, there is an unmet need in the field for the development of safe and efficacious treatment regimens for the treatment of HER2-positive cancers.

### SUMMARY OF THE INVENTION

The present invention relates to methods of treating a subject having a HER2-positive cancer using HER2-targeted T cell-dependent bispecific (TDB) antibodies.

In one aspect, the invention provides a method of treating or delaying the progression of a HER2-positive cancer in a subject in need thereof, the method comprising administering to the subject a treatment regimen comprising a HER2 antibody (e.g., a HER2 antibody that is not a HER2 T cell-dependent antibody (TDB), such as a monospecific HER2 antibody, e.g., a monospecific, bivalent HER2 antibody, e.g., trastuzumab) and a HER2 TDB comprising an anti-HER2 arm and an anti-CD3 arm (e.g., BTRC4017A), wherein the HER2 antibody and the HER2 TDB both bind domain IV of HER2, and wherein the treatment regimen results in an increased therapeutic index of the HER2 TDB as compared to treatment with the HER2 TDB in the absence of the HER2 antibody. In some embodiments, the increased therapeutic index is associated with a decreased likelihood of experiencing an on-target/off-tumor effect as compared to treatment with the HER2 TDB in the absence of the HER2 antibody. In some embodiments, the on-target/off-tumor effect is a symptom of pulmonary toxicity (e.g., interstitial lung disease, acute respiratory distress syndrome, dyspnea, cough, fatigue, and pulmonary infiltrates), an elevated liver enzyme level, dry mouth, dry eyes, mucositis, esophagitis, or a urinary symptom. In some embodiments, the increased therapeutic index is associated with a decreased likelihood of experiencing an immunogenic side effect as compared to treatment with the HER2 TDB in the absence of the HER2 antibody. The immunogenic side effect may include, e.g., an elevated level of anti-drug antibodies, an infusion/administration-related reaction (ARR), cardiac dysfunction, a pulmonary reaction, or cytokine release syndrome (CRS).

In some embodiments, the HER2 TDB and the HER2 antibody bind competitively to domain IV of HER2. In some embodiments, the HER2 antibody comprises: (i) a complementarity-determining region (CDR)-H1 comprising the amino acid sequence of SEQ ID NO: 1; (ii) a CDR-H2 comprising the amino acid sequence of SEQ ID NO: 2; (iii) a CDR-H3 comprising the amino acid sequence of SEQ ID NO: 3; (iv) a CDR-L1 comprising the amino acid sequence of SEQ ID NO: 4; (v) a CDR-L2 comprising the amino acid sequence of SEQ ID NO: 5; and (vi) a CDR-L3 comprising the amino acid sequence of SEQ ID NO: 6. In some embodiments, the HER2 antibody comprises a variable heavy chain domain (V_{H}) comprising at least 95% sequence identity (e.g., at least 96%, 97%, 98%, 99%, or 100% sequence identity) to the amino acid sequence of SEQ ID NO: 7 and/or a variable light chain domain (V_{L}) comprising at least 95% sequence identity (e.g., at least 96%, 97%, 98%, 99%, or 100% sequence identity) to the amino acid sequence of SEQ ID NO: 8. In particular embodiments, the V_{H} comprises the amino acid sequence of SEQ ID NO: 7 and/or the V_{L} comprises the amino acid sequence of SEQ ID NO: 8.

In some embodiments, the HER2 antibody (e.g., the additional HER2 antibody that is not a HER2 TDB) is monospecific and/or bivalent to HER2. In some embodiments, the HER2 antibody is a full-length antibody comprising an Fc region (e.g., trastuzumab). In some embodiments, the HER2 antibody is an Fc-modified trastuzumab variant, e.g., an Fc-modified trastuzumab variant having one or more amino acid modifications that reduces effector function (e.g., one or more substitution mutations, e.g., at amino acid residue L234, L235, and/or P329 (EU numbering). For example, in some embodiments, the one or more amino acid modifications comprise the substitution mutations L234A, L235A, and P329G (LALAPG).

In some embodiments of any of the preceding methods, the anti-HER2 arm of the HER2 TDB comprises a HER2 binding domain comprising: (i) a CDR-H1 comprising the amino acid sequence of SEQ ID NO: 1; (ii) a CDR-H2 comprising the amino acid sequence of SEQ ID NO: 2; (iii) a CDR-H3 comprising the amino acid sequence of SEQ ID NO: 3; (iv) a CDR-L1 comprising the amino acid sequence of SEQ ID NO: 4; (v) a CDR-L2 comprising the amino acid sequence of SEQ ID NO: 5; and (vi) a CDR-L3 comprising the amino acid sequence of SEQ ID NO: 6. In some embodiments, the HER2 binding domain comprises a V_{H} comprising at least 95% sequence identity (e.g., at least 96%, 97%, 98%, 99%, or 100% sequence identity) to the amino acid sequence of SEQ ID NO: 7 and/or a V_{L} comprising at least 95% sequence identity (e.g., at least 96%, 97%, 98%, 99%, or 100% sequence identity) to the amino acid sequence of SEQ ID NO: 8. In some embodiments, the V_{H} of the HER2 binding domain comprises the amino acid sequence of SEQ ID NO: 7 and/or the V_{L} of the HER2 binding domain comprises the amino acid sequence of SEQ ID NO: 8.

In some embodiments, the anti-CD3 arm of the HER2 TDB comprises a CD3 binding domain comprising: (i) a CDR-H1 comprising the amino acid sequence of SEQ ID NO: 9; (ii) a CDR-H2 comprising the amino acid sequence of SEQ ID NO: 10; (iii) a CDR-H3 comprising the amino acid sequence of SEQ ID NO: 11; (iv) a CDR-L1 comprising the amino acid sequence of SEQ ID NO: 12; (v) a CDR-L2 comprising the amino acid sequence of SEQ ID NO: 13; and (vi) a CDR-L3 comprising the amino acid sequence of SEQ ID NO: 14. In some embodiments, the CD3 binding domain comprises a V_{H} comprising at least 95% sequence identity (e.g., at least 96%, 97%, 98%, 99%, or 100% sequence identity) to the amino acid sequence of SEQ ID NO: 15 and/or a variable V_{L} comprising at least 95% sequence identity (e.g., at least 96%, 97%, 98%, 99%, or 100% sequence identity) to the amino acid sequence of SEQ ID NO: 16. In some embodiments, the V_{H} of the CD3 binding domain comprises the amino acid sequence of SEQ ID NO: 15 and/or the V_{L} of the CD3 binding domain comprises the amino acid sequence of SEQ ID NO: 16.

In some embodiments, (i) the anti-HER2 arm of the HER2 TDB comprises a HER2 binding domain comprising (a) a V_{H} comprising an amino acid sequence of SEQ ID NO: 7 and (b) a V_{L} comprising an amino acid sequence of SEQ ID NO: 8, and (ii) the anti-CD3 arm of the HER2 TDB comprises a CD3 binding domain comprising (a) a V_{H} comprising an amino acid sequence of SEQ ID NO: 15 and (b) a V_{L} comprising an amino acid sequence of SEQ ID NO: 16. In some embodiments, the HER2 TDB is BTRC4017A.

In some embodiments of any of the methods described herein, the HER2 TDB is a full-length antibody comprising a modified Fc region. The modified Fc region can include one or more substitution mutations that reduces effector function of the HER2 TDB. In some embodiments, the one or more substitution mutations comprise mutations at amino acid residues L234, L235, and/or D265 (EU numbering). In some embodiments, the one or more substitution mutations are L234A, L235A, and D265A. Additionally or alternatively, the one or more substitution mutations comprise an aglycosylation site mutation (e.g., an aglycosylation site mutation at amino acid residue N297 (EU numbering), e.g., an aglycosylation site mutation of N297G or N297A. In some embodiments, the modified Fc region comprises N297G, L234A, L235A, and D265A substitution mutations. In some embodiments, the HER2 TDB comprises one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, second CH2 (CH2₂) domain, and a second CH3 (CH3₂) domain. In some embodiments, at least one of the one or more heavy chain constant domains is paired with another heavy chain constant domain, wherein: (i) the CH3₁ and CH3₂ domains each comprise a protuberance or cavity, and wherein the protuberance or cavity in the CH3₁ domain is positionable in the cavity or protuberance, respectively, in the CH3₂ domain; or (ii) the CH2₁ and CH2₂ domains each comprise a protuberance or cavity, and wherein the protuberance or cavity in the CH2₁ domain is positionable in the cavity or protuberance, respectively, in the CH2₂domain.

In another aspect, the invention provides a method of treating or delaying the progression of a HER2-positive cancer (e.g., a HER2-positive breast cancer or a HER2-positive gastric cancer) in a subject in need thereof, the method comprising administering to the subject a treatment regimen comprising a HER2 antibody and a HER2 TDB, wherein (a) the HER2 antibody is trastuzumab or an Fc-modified trastuzumab variant, and (b) the HER2 TDB comprises an anti-HER2 arm and an anti-CD3 arm, wherein the anti-HER2 arm comprises a HER2 binding domain comprising: (i) a CDR-H1 comprising the amino acid sequence of SEQ ID NO: 1; (ii) a CDR-H2 comprising the amino acid sequence of SEQ ID NO: 2; (iii) a CDR-H3 comprising the amino acid sequence of SEQ ID NO: 3; (iv) a CDR-L1 comprising the amino acid sequence of SEQ ID NO: 4; (v) a CDR-L2 comprising the amino acid sequence of SEQ ID NO: 5; and (vi) a CDR-L3 comprising the amino acid sequence of SEQ ID NO: 6; and wherein the anti-CD3 arm comprises a CD3 binding domain comprising: (i) a CDR-H1 comprising the amino acid sequence of SEQ ID NO: 9; (ii) a CDR-H2 comprising the amino acid sequence of SEQ ID NO: 10; (iii) a CDR-H3 comprising the amino acid sequence of SEQ ID NO: 11; (iv) a CDR-L1 comprising the amino acid sequence of SEQ ID NO: 12; (v) a CDR-L2 comprising the amino acid sequence of SEQ ID NO: 13; and (vi) a CDR-L3 comprising the amino acid sequence of SEQ ID NO: 14; wherein the treatment regimen results in an increased therapeutic index of the HER2 TDB as compared to treatment with the HER2 TDB in the absence of the HER2 antibody. The increased therapeutic index may be associated with a decreased likelihood of experiencing an on-target/off-tumor effect as compared to treatment with the HER2 TDB in the absence of the HER2 antibody. In some embodiments, the on-target/off-tumor effect is a symptom of pulmonary toxicity (e.g., interstitial lung disease, acute respiratory distress syndrome, dyspnea, cough, fatigue, and pulmonary infiltrates), an elevated liver enzyme level, dry mouth, dry eyes, mucositis, esophagitis, or a urinary symptom. In some embodiments, the increased therapeutic index is associated with a decreased likelihood of experiencing an immunogenic side effect as compared to treatment with the HER2 TDB in the absence of the HER2 antibody. The immunogenic side effect may include, e.g., an elevated level of anti-drug antibodies, an infusion/administration-related reaction (ARR), cardiac dysfunction, a pulmonary reaction, or cytokine release syndrome (CRS).

In some embodiments of either of the aforementioned aspects, the HER2 antibody is administered prior to administration of the HER2 TDB.

In some embodiments, the HER2 antibody is administered at a dose of about 5 mg/kg to about 10 mg/kg (e.g., 5 mg/kg to 10 mg/kg or 6 mg/kg to 8 mg/kg, e.g., about 5 mg/kg, about 6 mg/kg, about 7 mg/kg, about 8 mg/kg, about 9 mg/kg, or about 10 mg/kg). In some embodiments, the HER2 antibody is administered about once every three weeks (Q3W).

In some embodiments, the HER2 TDB is administered at a fixed dose of 0.001 mg to 500 mg (e.g., from 0.003 mg to 250 mg, from 0.005 mg to 200 mg, from 0.01 mg to 150 mg, from 0.05 mg to 120 mg, from 0.1 mg to 100 mg, from 0.5 mg to 80 mg, or from 1.0 mg to 50 mg, e.g., from 0.001 mg to 0.005 mg, from 0.005 mg to 0.01 mg, from 0.01 mg to 0.05 mg, from 0.05 mg to 0.1 mg, from 0.1 mg to 0.5 mg, from 0.5 mg to 1.0 mg, from 1.0 mg to 5 mg, from 5 mg to 10 mg, from 10 mg to 20 mg, from 20 mg to 30 mg, from 30 mg to 40 mg, from 40 mg to 50 mg, from 50 mg to 60 mg, from 60 mg to 70 mg, from 70 mg to 80 mg, from 80 mg to 90 mg, from 90 mg to 100 mg, from 100 mg to 120 mg, from 120 mg to 150 mg, from 150 mg to 200 mg, from 200 mg to 250 mg, from 250 mg to 300 mg, from 300 mg to 350 mg, from 350 mg to 400 mg, from 400 mg to 450 mg, or from 450 mg to 500 mg, e.g., about 0.003 mg, about 0.005 mg, about 0.01 mg, about 0.05 mg, about 0.1 mg, about 0.5 mg, about 1.0 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, or about 250 mg, e.g., 0.003 mg, 0.009 mg, 0.027 mg, 0.081 mg, 0.24 mg, 0.72 mg, 1.08 mg, 1.51 mg, 2.2 mg, 2.3 mg, 4.0 mg, 4.6 mg, 6.6 mg, 8.0 mg, 9.2 mg, 12 mg, 13.2 mg, 14.8 mg, 18.4 mg, 19.8 mg, 26.4 mg, 36.8 mg, 51.5 mg, 52.8 mg, 61.3 mg, 72.1 mg, 105.6 mg, 147.8 mg, 176 mg, or 207 mg). In some embodiments, the HER2 TDB is administered about once every three weeks (Q3W).

In some embodiments of any of the methods described above, the treatment regimen comprises: (a) a first dose of the HER2 antibody; (b) a first dosing cycle (C1) after the first dose of the HER2 antibody, the C1 comprising a first dose of the HER2 TDB (C1D1) and a second dose of the HER2 TDB (C1D2), wherein the C1D2 is greater than the C1D1; (c) a second dosing cycle (C2) after the C1, the C2 comprising: (i) a second dose of the HER2 antibody; and (ii) an additional dose of the HER2 TDB (C2D1) after the second dose of the HER2 antibody, wherein the C2D1 is equivalent to the highest dose of the HER2 TDB of the C1.

In another aspect of the invention, provided herein is a method of treating or delaying the progression of a HER2-positive cancer in a subject in need thereof, the method comprising administering to the subject a treatment regimen comprising a HER2 antibody and a HER2 TDB, wherein the HER2 TDB comprises an anti-HER2 arm and an anti-CD3 arm, wherein the HER2 antibody and the HER2 TDB both bind domain IV of HER2, wherein the treatment regimen comprises: (a) a first dose of the HER2 antibody; (b) a first dosing cycle (C1) after the first dose of the HER2 antibody, the C1 comprising a first dose of the HER2 TDB (C1D1) and a second dose of the HER2 TDB (C1D2), wherein the C1D2 is greater than the C1D1; (c) a second dosing cycle (C2) after the C1, the C2 comprising: (i) a second dose of the HER2 antibody; and (ii) an additional dose of the HER2 TDB (C2D1) after the second dose of the HER2 antibody, wherein the C2D1 is equivalent to the highest dose of the HER2 TDB of the C1.

In some embodiments, the first dose of the HER2 antibody is administered one day prior to the C1D1, and wherein the subject is monitored for a period of 30 minutes to 24 hours (e.g., 30 minutes to 2 hours, e.g., 30 minutes to 90 minutes, e.g., 30 minutes, 60 minutes, 90 minutes, or 120 minutes) between the first dose of the HER2 antibody and the C1D1.

In some embodiments, the first dose of the HER2 antibody is from 5 mg/kg to 10 mg/kg (e.g., about 6 mg/kg or about 8 mg/kg). In some embodiments, the first dose of the HER2 antibody is 6 mg/kg. In other embodiments, the first dose of the HER2 antibody is 8 mg/kg. In some embodiments, the second dose of the HER2 antibody is from 5 mg/kg to 10 mg/kg (e.g., about 6 mg/kg). In some embodiments, the second dose of the HER2 antibody is 6 mg/kg. In some embodiments, the first and/or second dose of the HER2 antibody is administered by infusion over a period of at least 30 minutes.

In some embodiments, the second dose of the HER2 antibody is administered on the same day as the C2D1. In some embodiments, the C1D2 is at least two-fold the dose of the C1D1 (e.g., at least three-fold the dose of the C1D1). In some embodiments, the C1D1 is from 0.003 mg to 50 mg (e.g., from 0.003 mg to 50 mg, from 0.005 mg to 20 mg, from 0.01 mg to 10 mg, from 0.05 mg to 8 mg, or from 0.1 mg to 5 mg, e.g., from 0.001 mg to 0.005 mg, from 0.005 mg to 0.01 mg, from 0.01 mg to 0.05 mg, from 0.05 mg to 0.1 mg, from 0.1 mg to 0.5 mg, from 0.5 mg to 1.0 mg, from 1.0 mg to 5 mg, from 5 mg to 10 mg, from 10 mg to 20 mg, from 20 mg to 30 mg, from 30 mg to 40 mg, or from 40 mg to 50 mg, e.g., about 0.003 mg, about 0.005 mg, about 0.01 mg, about 0.05 mg, about 0.1 mg, about 0.5 mg, about 1.0 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 30 mg, about 35 mg, about 40 mg, about 45 mg, or about 50 mg). In some embodiments, the C1D1 is 0.003 mg, 0.009 mg, 0.027 mg, 0.081 mg, 0.12 mg, 0.24 mg, 0.48 mg, 0.72 mg, 1.0 mg, 2.0 mg, 2.2 mg, 4.0 mg, 6.6 mg, 8.0 mg, 12 mg, 18 mg, 27 mg, or 40.5 mg.

In some embodiments, the C1D2 is from 0.009 mg to 200 mg (e.g., from 0.01 mg to 150 mg, from 0.05 mg to 100 mg, from 0.1 mg to 50 mg, from 0.5 mg to 20 mg, or from 1 mg to 10 mg, e.g., from 0.009 mg to 0.01 mg, from 0.01 mg to 0.05 mg, from 0.05 mg to 0.1 mg, from 0.1 mg to 0.5 mg, from 0.5 mg to 1.0 mg, from 1.0 mg to 5 mg, from 5 mg to 10 mg, from 10 mg to 20 mg, from 20 mg to 30 mg, from 30 mg to 40 mg, from 40 mg to 50 mg, from 50 mg to 60 mg, from 60 mg to 70 mg, from 70 mg to 80 mg, from 80 mg to 90 mg, from 90 mg to 100 mg, from 100 mg to 120 mg, from 120 mg to 150 mg, or from 150 mg to 200 mg, e.g., about 0.009 mg, about 0.01 mg, about 0.05 mg, about 0.1 mg, about 0.5 mg, about 1.0 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, or about 200 mg). In some embodiments, the C1D2 is 0.009 mg, 0.027 mg, 0.081 mg, 0.24 mg, 0.4 mg, 0.72 mg, 0.08 mg, 1.6 mg, 2.2 mg, 2.3 mg, 3.2 mg, 4.6 mg, 6.4 mg, 6.6 mg, 9.2 mg, 12.8 mg, 14.8 mg, 18.4 mg, 19.8 mg, 25.6 mg, 36.8 mg, 38.4, 51.5 mg, 57.6 mg, 72.1 mg, 86.4 mg, 61.3 mg, or 129.6 mg.

In some embodiments, e.g., in a one-step fractionation, the C2D1 and the C1D2 are equivalent.

In some embodiments, the C1 further comprises a third dose of the HER2 TDB (C1D3), wherein the C1D3 is greater than the C1D2. In some embodiments, the C1D1, the C1D2, and the C1D3 are cumulatively greater than a highest cleared dose of the HER2 TDB in a first dosing cycle of a one-step fractionation, dose-escalation dosing regimen (e.g., where the highest cleared dose is between about 0.01 mg and about 30 mg, e.g., from 0.5 mg to 25 mg, from 1 mg to 20 mg, or from 2 mg to 10 mg). In some embodiments, the C1D2 is from two-fold to ten-fold (e.g., about two-fold, about three-fold, about four-fold, about five-fold, about six-fold, about seven-fold, about eight-fold, about nine-fold, or about ten-fold) the dose of the C1D1. In some embodiments, the C1D3 is from two-fold to three-fold the dose of the C1D2. In some embodiments, the C2D1 and the C1D3 are equivalent.

In some embodiments, the C1D1 is from 0.01 mg to 20 mg (e.g., from 0.05 mg to 15 mg, from 0.1 mg to 10 mg, or from 0.5 mg to 5 mg, e.g., from 0.01 mg to 0.05 mg, from 0.05 mg to 0.1 mg, from 0.1 mg to 0.5 mg, from 0.5 mg to 1.0 mg, from 1.0 mg to 5 mg, from 5 mg to 10 mg, from 10 mg to 15 mg, or from 15 mg to 20 mg, e.g., about 0.01 mg, about 0.05 mg, about 0.1 mg, about 0.5 mg, about 1.0 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg).

In some embodiments, the C1D2 is from 0.1 mg to 100 mg (e.g., from 0.1 mg to 80 mg, from 0.5 mg to 50 mg, or from 1 mg to 10 mg, e.g., from 0.1 mg to 0.5 mg, from 0.5 mg to 1.0 mg, from 1.0 mg to 5 mg, from 5 mg to 10 mg, from 10 mg to 20 mg, from 20 mg to 30 mg, from 30 mg to 40 mg, from 40 mg to 50 mg, from 50 mg to 60 mg, from 60 mg to 70 mg, from 70 mg to 80 mg, from 80 mg to 90 mg, or from 90 mg to 100 mg, e.g., about 0.01 mg, about 0.05 mg, about 0.1 mg, about 0.5 mg, about 1.0 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg).

In some embodiments, the C1D3 is from 1 mg to 400 mg (e.g., from 10 mg to 300 mg, from 20 mg to 200 mg, or from 50 mg to 100 mg, e.g., from 1.0 mg to 5 mg, from 5 mg to 10 mg, from 10 mg to 20 mg, from 20 mg to 30 mg, from 30 mg to 40 mg, from 40 mg to 50 mg, from 50 mg to 60 mg, from 60 mg to 70 mg, from 70 mg to 80 mg, from 80 mg to 90 mg, from 90 mg to 100 mg, from 100 mg to 120 mg, from 120 mg to 150 mg, from 150 mg to 200 mg, from 200 to 250 mg, from 250 mg to 300 mg, from 300 mg to 350 mg, or from 350 mg to 400 mg, e.g., about 1.0 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, or about 400 mg). In some embodiments, the C1D3 is 1.1 mg, 2.2 mg, 4.4 mg, 6.6 mg, 8.8 mg, 13.2 mg, 17.6 mg, 26.4 mg, 35.2 mg, 52.8 mg, 70.4 mg, 105.6 mg, 147.8 mg, 158.4 mg, 176 mg, 207 mg, 237.6 mg, or 356.4 mg.

In some embodiments, the method comprises administering to the subject the C1D1, the C1D2, and the C1D3 on or about Days 1, 8, and 15, respectively, of the C1. In some embodiments, the C1 is about 21 days. In some embodiments, the C2 is about 21 days.

In some embodiments, the method comprises administering to the subject the C2D1 on Day 1 of the C2. In some embodiments, the treatment regimen comprises one or more additional dosing cycles (e.g., up to 15 additional dosing cycles, e.g., one, two, three, four, five, six, seven eight, nine, ten, eleven, twelve, thirteen, fourteen, or fifteen additional dosing cycles). In some embodiments, the length of each of the one or more additional dosing cycles is about 21 days. In some embodiments, each of the one or more additional dosing cycles comprises a single dose of the HER2 antibody and a single dose of the HER2 TDB (e.g., wherein the HER2 antibody is administered prior to the HER2 TDB on each of the additional dosing cycles, e.g., on Day 1 of each of the additional dosing cycles). In some embodiments, the method comprises administering to the subject the HER2 antibody and the HER2 TDB on Day 1 of each of the one or more additional dosing cycles.

In another aspect, the invention provides a method of treating or delaying the progression of a HER2-positive cancer in a subject in need thereof, the method comprising administering to the subject a treatment regimen comprising a HER2 TDB, wherein the treatment regimen comprises: (a) a first cycle (C1) comprising a first dose of the HER2 TDB (C1D1) and a second dose of the HER2 TDB (C1D2), wherein the C1D2 is greater than the C1D1; and (b) a second cycle (C2) comprising an additional dose of the HER2 TDB (C2D1), wherein the C2D1 is equivalent to the highest dose of the HER2 TDB of the C1. In some embodiments, the C1D2 is at least two-fold the dose of the C1D1 (e.g., at least three-fold the dose of the C1D1).

In some embodiments, the C1D1 is from 0.003 mg to about 10 mg (e.g., from 0.005 mg to 9 mg, from 0.01 mg to 8 mg, from 0.05 mg to 7 mg, or from 0.1 mg to 5 mg, e.g., from 0.003 mg to 0.005 mg, from 0.005 mg to 0.01 mg, from 0.01 mg to 0.05 mg, from 0.05 mg to 0.1 mg, from 0.1 mg to 0.5 mg, from 0.5 mg to 1.0 mg, from 1.0 mg to 5 mg, or from 5 mg to 10 mg, e.g., about 0.003 mg, about 0.005 mg, about 0.01 mg, about 0.05 mg, about 0.1 mg, about 0.5 mg, about 1.0 mg, about 2 mg, about 3 mg, about 4 mg, about 5 mg, about 6 mg, about 7 mg, about 8 mg, about 9 mg, or about 10 mg).

In some embodiments, the C1D2 is from 0.009 to about 20 mg (e.g., from 0.01 mg to 15 mg, from 0.05 mg to 10 mg, or from 0.1 mg to 5 mg, e.g., from 0.009 mg to 0.01 mg, from 0.01 mg to 0.05 mg, from 0.05 mg to 0.1 mg, from 0.1 mg to 0.5 mg, from 0.5 mg to 1.0 mg, from 1.0 mg to 5 mg, from 5 mg to 10 mg, from 10 mg to 15 mg, or from 15 mg to 20 mg, e.g., about 0.009 mg, about 0.01 mg, about 0.05 mg, about 0.1 mg, about 0.5 mg, about 1.0 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg).

In some embodiments, the C2D1 and the C1D2 are equivalent. In other embodiments, the C1 further comprises a third dose of the HER2 TDB (C1D3) which is greater than the C1D2. In some embodiments, the C1D1, the C1D2, and the C1D3 are cumulatively greater than a highest cleared dose of the HER2 TDB in a first dosing cycle of a one-step fractionation, dose-escalation dosing regimen. In some embodiments, the highest cleared dose is between about 0.01 mg and about 30 mg. In some embodiments, the C1D2 is from two-fold to ten-fold the dose of the C1D1 (e.g., about three-fold, about four-fold, about five-fold, about six-fold, about seven-fold, about eight-fold, about nine-fold, or about ten-fold the dose of the C1D1). In some embodiments, the C1D3 is from two-fold to three-fold the dose of the C1D2. In some embodiments, the C2D1 and the C1D3 are equivalent.

In some embodiments, the C1D1 is from 0.01 mg to 20 mg (e.g., from 0.01 mg to 15 mg, from 0.05 mg to 10 mg, or from 0.1 mg to 5 mg, e.g., from 0.01 mg to 0.05 mg, from 0.05 mg to 0.1 mg, from 0.1 mg to 0.5 mg, from 0.5 mg to 1.0 mg, from 1.0 mg to 5 mg, from 5 mg to 10 mg, from 10 mg to 15 mg, or from 15 mg to 20 mg, e.g., about 0.01 mg, about 0.05 mg, about 0.1 mg, about 0.5 mg, about 1.0 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg).

In some embodiments, the C1D2 is from 0.1 mg to 100 mg (e.g., from 0.1 mg to 80 mg, from 0.5 mg to 50 mg, or from 1 mg to 10 mg, e.g., from 0.1 mg to 0.5 mg, from 0.5 mg to 1.0 mg, from 1.0 mg to 5 mg, from 5 mg to 10 mg, from 10 mg to 20 mg, from 20 mg to 30 mg, from 30 mg to 40 mg, from 40 mg to 50 mg, from 50 mg to 60 mg, from 60 mg to 70 mg, from 70 mg to 80 mg, from 80 mg to 90 mg, or from 90 mg to 100 mg, e.g., about 0.01 mg, about 0.05 mg, about 0.1 mg, about 0.5 mg, about 1.0 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg).

In some embodiments, the C1D3 is from 1 mg to 200 mg (e.g., from 10 mg to 150 mg, from 20 mg to 120 mg, or from 50 mg to 100 mg, e.g., from 1.0 mg to 5 mg, from 5 mg to 10 mg, from 10 mg to 20 mg, from 20 mg to 30 mg, from 30 mg to 40 mg, from 40 mg to 50 mg, from 50 mg to 60 mg, from 60 mg to 70 mg, from 70 mg to 80 mg, from 80 mg to 90 mg, from 90 mg to 100 mg, from 100 mg to 120 mg, from 120 mg to 150 mg, or from 150 mg to 200 mg, e.g., about 1.0 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, or about 200 mg).

In some embodiments, the method comprises administering to the subject the C1D1, the C1D2, and the C1D3 on or about Days 1, 8, and 15, respectively, of the C1. In some embodiments, the C1 is about 21 days. Additionally or alternatively, in some embodiments, the C2 is 21 days. In some embodiments, the method comprises administering to the subject the C2D1 on Day 1 of the C2. The treatment regimen may include one or more additional dosing cycles (e.g., up to 15 additional dosing cycles, e.g., one, two, three, four, five, six, seven eight, nine, ten, eleven, twelve, thirteen, fourteen, or fifteen additional dosing cycles). In some embodiments, each of the additional dosing cycles is about 21 days. In some embodiments, each of the additional dosing cycles comprises a single dose of the HER2 TDB. In some embodiments, the method comprises administering to the subject the HER2 TDB on Day 1 of each of the one or more additional dosing cycles. In some embodiments of any of the preceding aspects, the HER2 antibody and/or the HER2 TDB are administered by intravenous infusion (e.g. by IV bag). In some embodiments, the treatment regimen results in an increased therapeutic index of the HER2 TDB as compared to a control treatment regimen (e.g., treatment with the HER2 TDB in the absence of the HER2 antibody, or a treatment regimen without fractionated dosing).

In some embodiments of any of the preceding aspect, the method further includes administering one or more additional therapeutic agents. For example, the one or more additional therapeutic agents can be tocilizumab, a corticosteroid, a PD-1 axis antagonist, or an antibody-drug conjugate. In some embodiments, the PD-1 axis binding antagonist is selected from the group consisting of a PD-L1 binding antagonist (e.g., MPDL3280A (atezolizumab), YW243.55.S70, MDX-1105, or MED14736), a PD-1 binding antagonist (e.g., MDX-1106 (nivolumab), MK-3475 (pembrolizumab), and AMP-224), and a PD-L2 binding antagonist (e.g., a PD-L1 binding antibody or an immunoadhesin).

In some embodiments, the subject has been administered trastuzumab in a previous treatment regimen (e.g. as a treatment for a HER2-positive cancer).

In some embodiments, the HER2-positive cancer is a HER2-positive solid tumor. Additionally or alternatively, the HER2-positive cancer can be a locally advanced or metastatic HER2-positive cancer. In some embodiments, the HER2-positive cancer is a HER2-positive breast cancer or a HER2-positive gastric cancer (e.g., a HER2-positive gastroesophageal junction cancer or a HER2-positive colorectal cancer. In some embodiments, the HER2-positive cancer is selected from the group consisting of a HER2-positive gastroesophageal junction cancer, a HER2-positive colorectal cancer, a HER2-positive lung cancer (e.g., a HER2-positive non-small cell lung carcinoma), a HER2-positive pancreatic cancer, a HER2-positive colorectal cancer, a HER2-positive bladder cancer, a HER2-positive salivary duct cancer, a HER2-positive ovarian cancer (e.g., a HER2-positive epithelial ovarian cancer), or a HER2-positive endometrial cancer.

### BRIEF DESCRIPTION OF THE DRAWINGS

FIG. 1 is a rendering of the crystal structure of the HER2 extracellular domain (ECD) bound by the HER2 binding domains 4D5 (trastuzumab), 2C4 (pertuzumab), and 7C2.

FIG. 2 is an immunoblot showing the relative expression of HER2 protein by MCF7 cells, HT55 cells, and KPL4 cells.

FIG. 3A is a graph showing relative killing of KPL4 cells by BTRC4017A alone (red circles); BTRC4017A+230 μg/mL trastuzumab (blue squares); and BTRC4017A+60 μg/mL trastuzumab (brown triangles) as a function of BTRC4017A concentration (ng/mL).

FIG. 3B is a graph showing relative killing of HT55 cells by BTRC4017A alone (red circles); BTRC4017A+230 μg/mL trastuzumab (blue squares); and BTRC4017A+60 μg/mL trastuzumab (brown triangles) as a function of BTRC4017A concentration (ng/mL). N.D.=Not determined.

FIG. 4 is a graph showing binding of trastuzumab (red circles) and trastuzumab-LALAPG (blue squares) to HER2-expressing SKBR3 cells as a function of concentration. Binding was detected using a goat anti-human-FITC secondary antibody, the presence of which was quantified by mean fluorescence intensity (MFI) using flow cytometry.

FIG. 5A is a trellis plot showing KPL4 tumor volume over the course of various treatments in a mouse model. The top row shows the effect of control treatments; the left-hand graph shows tumor growth in response to vehicle administration; the middle graph shows tumor growth in response to trastuzumab (HERCEPTIN®) without peripheral blood mononuclear cells (PBMCs); and the right-hand graph shows tumor growth in response to trastuzumab (HERCEPTIN®) with PBMCs. The middle row and bottom row show tumor growth over the course of treatment with BTRC4017A alone and in combination with trastuzumab (HERCEPTIN®), respectively. In the middle row and bottom row, the left-hand graph shows tumor growth in response to 0.05 mg/kg BTRC4017A; the middle graph shows tumor growth in response to 0.5 mg/kg BTRC4017A; and the right-hand graph shows tumor growth in response to 5.0 mg/kg BTRC4017A. Bold, solid lines represent the fitted tumor volume for each group. Dashed lines represent the fitted tumor volume for the vehicle control group. Gray lines represent individual animals.

FIG. 5B is a trellis plot showing HT55 tumor volume over the course of various treatments in a mouse model. The top row shows the effect of control treatments; the left-hand graph shows tumor growth in response to vehicle administration; the middle graph shows tumor growth in response to trastuzumab (HERCEPTIN®) without peripheral blood mononuclear cells (PBMCs); and the right-hand graph shows tumor growth in response to trastuzumab (HERCEPTIN®) with PBMCs. The middle row and bottom row show tumor growth over the course of treatment with BTRC4017A alone and in combination with trastuzumab (HERCEPTIN®), respectively. In the middle row and bottom row, the left-hand graph shows tumor growth in response to 0.05 mg/kg BTRC4017A; the middle graph shows tumor growth in response to 0.5 mg/kg BTRC4017A; and the right-hand graph shows tumor growth in response to 5.0 mg/kg BTRC4017A. Bold, solid lines represent the fitted tumor volume for each group. Dashed lines represent the fitted tumor volume for the vehicle control group. Gray lines represent individual animals.

### DETAILED DESCRIPTION OF EMBODIMENTS OF THE INVENTION

### I. Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

The term “about” as used herein refers to the usual error range for the respective value readily known to the skilled person in this technical field. Reference to “about” a value or parameter herein includes (and describes) embodiments that are directed to that value or parameter per se.

As used herein, the singular forms “a,” “an,” and “the” include plural referents unless the context clearly dictates otherwise. For example, reference to “an isolated peptide” means one or more isolated peptides.

Throughout this specification and claims, the word “comprise,” or variations such as “comprises” or “comprising” will be understood to imply the inclusion of a stated integer or group of integers but not the exclusion of any other integer or group of integers.

The term “antibody” herein is used in the broadest sense and encompasses various antibody structures, including but not limited to monoclonal antibodies, polyclonal antibodies, multispecific antibodies (e.g., bispecific antibodies), and antibody fragments so long as they exhibit the desired antigen-binding activity.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂; diabodies; linear antibodies; single-chain antibody molecules (e.g. scFv); and multispecific antibodies formed from antibody fragments.

By “antigen-binding moiety” is meant a part of a compound or a molecule that specifically binds to a target epitope, antigen, ligand, or receptor. Molecules featuring antigen-binding moieties include, but are not limited to, antibodies (e.g., monoclonal, polyclonal, recombinant, humanized, and chimeric antibodies), antibody fragments or portions thereof (e.g., Fab fragments, Fab′2, scFv antibodies, SMIP, domain antibodies, diabodies, minibodies, scFv-Fc, affibodies, nanobodies, and VH and/or VL domains of antibodies), receptors, ligands, aptamers, and other molecules having an identified binding partner. An “affinity matured” antibody refers to an antibody with one or more alterations in one or more hypervariable regions (HVRs), compared to a parent antibody which does not possess such alterations, such alterations resulting in an improvement in the affinity of the antibody for antigen.

By “binding domain” is meant a part of a compound or a molecule that specifically binds to a target epitope, antigen, ligand, or receptor. Binding domains can be part of a molecule such as an antibody (e.g., a monoclonal, polyclonal, recombinant, humanized, or chimeric antibody), an antibody fragment or portion thereof (e.g., a Fab fragment, Fab′2, scFv antibody, SMIP, domain antibody, diabody, minibody, scFv-Fc, affibody, nanobody and a VH and/or VL domain of an antibody), receptor, ligand, aptamer, or other molecule having an identified binding partner.

As used herein, the term “Complementarity Determining Regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined by Kabat (i.e. about residues 24-34 (L1), 50-56 (L2) and 89-97 (L3) in the light chain variable domain (V_{L}) and 31-35 (H1), 50-65 (H2) and 95-102 (H3) in the heavy chain variable domain (V_{H}); Kabat et al., Sequences of Proteins of Immunological Interest, 5th Ed. Public Health Service, National Institutes of Health, Bethesda, Md. (1991)) and/or those residues from a “hypervariable loop” (i.e., about residues 26-32 (L1), 50-52 (L2) and 91-96 (L3) in the light chain variable domain (V_{L}) and 26-32 (H1), 53-55 (H2) and 96-101 (H3) in the heavy chain variable domain (V_{H}); Chothia and Lesk J. Mol. Biol. 196:901-917 (1987)). In some instances, a complementarity determining region can include amino acids from both a CDR region defined according to Kabat and a hypervariable loop. For example, the CDRH1 of the heavy chain of antibody 4D5 includes amino acids 26 to 35.

The term “Fc region” herein is used to define a C-terminal region of an immunoglobulin heavy chain that contains at least a portion of the constant region. The term includes native sequence Fc regions and variant Fc regions. In one embodiment, a human IgG heavy chain Fc region extends from Cys226, or from Pro230, to the carboxyl-terminus of the heavy chain. However, the C-terminal lysine (Lys447) of the Fc region may or may not be present. Unless otherwise specified herein, numbering of amino acid residues in the Fc region or constant region is according to the EU numbering system, also called the EU index, as described in Kabat et al., supra.

The terms “full-length antibody,” “intact antibody,” and “whole antibody” are used herein interchangeably to refer to an antibody having a structure substantially similar to a native antibody structure or having heavy chains that contain an Fc region as defined herein.

“Effector functions” refer to those biological activities attributable to the Fc region of an antibody, which vary with the antibody isotype. Examples of antibody effector functions include: C1q binding and complement dependent cytotoxicity (CDC); Fc receptor binding; antibody-dependent cell-mediated cytotoxicity (ADCC); phagocytosis; down regulation of cell surface receptors (e.g. B cell receptor); and B cell activation.

“Framework” or “FR” refers to variable domain residues other than hypervariable region (HVR) residues. The FR of a variable domain generally consists of four FR domains: FR1, FR2, FR3, and FR4. Accordingly, the CDR and FR sequences generally appear in the following sequence in VH (or VL): FR1-H1(L1)-FR2-H2(L2)-FR3-H3(L3)-FR4.

“Percent (%) amino acid sequence identity” or “percent (%) sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for aligning sequences, including any algorithms needed to achieve maximal alignment over the full length of the sequences being compared. For purposes herein, however, % amino acid sequence identity values are generated using the sequence comparison computer program ALIGN-2. The ALIGN-2 sequence comparison computer program was authored by Genentech, Inc., and the source code has been filed with user documentation in the U.S. Copyright Office, Washington D.C., 20559, where it is registered under U.S. Copyright Registration No. TXU510087. The ALIGN-2 program is publicly available from Genentech, Inc., South San Francisco, Calif., or may be compiled from the source code. The ALIGN-2 program should be compiled for use on a UNIX operating system, including digital UNIX V4.0D. All sequence comparison parameters are set by the ALIGN-2 program and do not vary.

In situations where ALIGN-2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

100 times the fraction X/Y

where X is the number of amino acid residues scored as identical matches by the sequence alignment program ALIGN-2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A. Unless specifically stated otherwise, all % amino acid sequence identity values used herein are obtained as described in the immediately preceding paragraph using the ALIGN-2 computer program.

The term “chimeric” antibody refers to an antibody in which a portion of the heavy and/or light chain is derived from a particular source or species, while the remainder of the heavy and/or light chain is derived from a different source or species.

The “class” of an antibody refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

A “subject,” a “patient,” an “individual” is a mammal. Mammals include, but are not limited to, domesticated animals (e.g., cows, sheep, cats, dogs, and horses), primates (e.g., humans and non-human primates such as monkeys), rabbits, and rodents (e.g., mice and rats). In certain embodiments, the subject, patient, or individual is a human.

The term “HER2-positive” cancer comprises cancer cells which have higher than normal levels of HER2. Examples of HER2-positive cancer include HER2-positive breast cancer and HER2-positive gastric cancer. In some embodiments, a HER2-positive cancer is selected from the group consisting of a HER2-positive gastroesophageal junction cancer, a HER2-positive colorectal cancer, a HER2-positive lung cancer (e.g., HER2-positive non-small cell lung carcinoma), a HER2-positive pancreatic cancer, a HER2-positive colorectal cancer, a HER2-positive bladder cancer, a HER2-positive salivary duct cancer, a HER2-positive ovarian cancer (e.g., HER2-positive epithelial ovarian cancer), or a HER2-positive endometrial cancer. In some embodiments, a HER2-positive cancer is locally advanced or metastatic. Optionally, HER2-positive cancer has an immunohistochemistry (IHC) score of 2+ or 3+ and/or an in situ hybridization (ISH) amplification ratio 2.0. In some embodiments, a HER2-positive breast cancer is defined according to the HER2 Testing in Breast Cancer Guideline: 2018 Focused Update (Wolff et al. J. Clin. Oncol. 2018, 36(20):2105-2122).

An “effective amount” of a compound, for example, a HER2 antibody (e.g., a HER2 TDB, trastuzumab, or the combination thereof), is at least the minimum amount required to achieve the desired therapeutic or prophylactic result, such as a measurable improvement or prevention of a particular disorder (e.g., a HER2-positive cancer, e.g., a HER2-positive breast cancer or a HER2-positive gastric cancer). An effective amount herein may vary according to factors such as the disease state, age, sex, and weight of the patient, and the ability of the antibody to elicit a desired response in the individual. An effective amount is also one in which any toxic or detrimental effects of the treatment are outweighed by the therapeutically beneficial effects. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells (e.g., HER2-positive cancer cells); reducing the tumor size; inhibiting (i.e., slow to some extent or desirably stop) cancer cell infiltration into peripheral organs; inhibit (i.e., slow to some extent and desirably stop) tumor metastasis; inhibiting to some extent tumor growth; and/or relieving to some extent one or more of the symptoms associated with the disorder. An effective amount can be administered in one or more administrations. For purposes of this invention, an effective amount of drug, compound, or pharmaceutical composition is an amount sufficient to accomplish prophylactic or therapeutic treatment either directly or indirectly. As is understood in the clinical context, an effective amount of a drug, compound, or pharmaceutical composition may or may not be achieved in conjunction with another drug, compound, or pharmaceutical composition. Thus, an “effective amount” may be considered in the context of administering one or more therapeutic agents, and a single agent may be considered to be given in an effective amount if, in conjunction with one or more other agents, a desirable result may be or is achieved.

The term “therapeutic index” refers to the ratio between a dose of therapeutic agent (e.g., a HER2 TDB, e.g., BTRC4017A) that elicits a toxic effect (e.g., an off-tumor toxic effect) and a dose of the therapeutic agent (e.g., the HER2 TDB, e.g., BTRC4017A) sufficient to achieve a desired therapeutic effect. An increased therapeutic index can be achieved when (a) the dose sufficient to achieve a desired therapeutic effect is decreased relative to a reference treatment regimen and/or (b) the dose at which the therapeutic agent elicits a toxic effect (e.g., maximum tolerated dose) is increased relative to a reference treatment regimen. In determining a therapeutic index, a dose sufficient to achieve a desired therapeutic effect can be determined according to a subject's objective response to the treatment regimen. In some embodiments, an objective response is a complete response (CR) or a partial response (PR) according to RECIST v.1.1. Additionally or alternatively, a dose sufficient to achieve a desired therapeutic effect can be determined according to a subject's duration of response (DOR). In some embodiments, a DOR is the time from the first occurrence of a documented objective response to the time of the first documented disease progression or death from any cause, whichever occurs first, per RECIST v.1.1. In determining a therapeutic index, a dose at which the therapeutic agent elicits a toxic effect can be determined according to the presence of a dose-limiting toxicity (DLT), which is graded according to National Cancer Institute Common Terminology Criteria for Adverse Events (NCI CTCAE) v5.0, with the exception of cytokine release syndrome (CRS), which is graded according to the Modified Cytokine Release Syndrome Grading System (see Tables 1 and 2 in Section II—Therapeutic Methods).

“Survival” refers to the subject remaining alive, and includes progression-free survival (PFS) and overall survival (OS). Survival can be estimated by the Kaplan-Meier method, and any differences in survival are computed using the stratified log-rank test.

“Progression-free survival (PFS)” refers to the time from treatment (or randomization) to first disease progression or death. For example it is the time that the subject remains alive, without return of the cancer, e.g., for a defined period of time such as about 1 month, 1.2 months, 2 months, 2.4 months, 2.9 months, 3 months, 3.5 months, 4, months, 6 months, 7 months, 8 months, 9 months, 1 year, about 2 years, about 3 years, etc., from initiation of treatment or from initial diagnosis. In one aspect of the invention, PFS can be assessed by Response Evaluation Criteria in Solid Tumors (RECIST v.1.1).

“Overall survival (OS)” refers to the subject remaining alive for a defined period of time, such as about 1 year, about 2 years, about 3 years, about 4 years, about 5 years, about 10 years, etc., from initiation of treatment or from initial diagnosis.

As used herein, the term “on-target/off-tumor effect” refers to an effect associated with binding by a therapeutic agent to a disease-associated target molecule that is expressed on a healthy cell (e.g., an effect associated with binding by a HER2 TDB (e.g., BTRC4017A) to a HER2 molecule expressed on a healthy cell, e.g., wherein the effect is a result of T cell cytotoxicity directed to the healthy cell). In some embodiments, an on-target/off-tumor effect is a symptom of pulmonary toxicity (e.g., interstitial lung disease, acute respiratory distress syndrome, dyspnea, cough, fatigue, or the presence of pulmonary infiltrates), an elevated liver enzyme level, dry mouth, dry eyes, mucositis, esophagitis, or a urinary symptom. Additionally or alternatively, an on-target/off-tumor effect can be any effect caused by aberrant function of a HER2-expressing healthy cell or tissue (i.e., non-cancerous cell or tissue), wherein the aberrant function is attributable to the administration of a HER2 antibody (e.g., a HER2 TDB antibody administered in the absence of an additional HER2 antibody (e.g., wherein the HER2 TDB antibody and the additional HER2 antibody bind domain IV of HER2). In some embodiments, an on-target/off-tumor effect is an immunogenic effect, such as CRS, which is graded according to the Modified Cytokine Release Syndrome Grading System (see Tables 1 and 2 in Section II—Therapeutic Methods).

The term “cluster of differentiation 3” or “CD3,” as used herein, refers to any native CD3 from any vertebrate source, including mammals such as primates (e.g. humans) and rodents (e.g., mice and rats), unless otherwise indicated, including, for example, CD3ε, CD3γ, CD3α, and CD3β chains. The term encompasses “full-length,” unprocessed CD3 (e.g., unprocessed or unmodified CD3ε or CD3γ), as well as any form of CD3 that results from processing in the cell. The term also encompasses naturally occurring variants of CD3, including, for example, splice variants or allelic variants. CD3 includes, for example, human CD3ε protein (NCBI RefSeq No. NP_000724), which is 207 amino acids in length, and human CD3γ protein (NCBI RefSeq No. NP_000064), which is 182 amino acids in length.

The term “HER2,” as used herein, refers to any native HER2 from any vertebrate source, including mammals such as primates (e.g. humans) and rodents (e.g., mice and rats), unless otherwise indicated. The term encompasses “full-length,” unprocessed HER2, as well as any form of HER2 that results from processing in the cell. The term also encompasses naturally occurring variants of HER2, including, for example, splice variants or allelic variants. HER2 includes, for example, human HER2 protein (see, e.g., NCBI RefSeq No. NP_001276865), which is 1240 amino acids in length. Domain IV of HER2 is the extracellular protein region that is positioned closest to the cellular membrane. Domain IV has the amino acid sequence of SEQ ID NO: 17.

As used herein, “treatment” (and grammatical variations thereof, such as “treat” or “treating”) refers to clinical intervention in an attempt to alter the natural course of the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies of the invention are used to delay development of a disease or to slow the progression of a disease.

As used herein, “delaying progression” of a disorder or disease means to defer, hinder, slow, retard, stabilize, and/or postpone development of the disease or disorder (e.g., a HER2-positive cancer, e.g., a HER2-positive breast cancer or a HER2-positive gastric cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

By “reduce” or “inhibit” is meant the ability to cause an overall decrease, for example, of 20% or greater, of 50% or greater, or of 75%, 85%, 90%, 95%, or greater. In certain embodiments, reduce or inhibit can refer to the reduction or inhibition of undesirable events (e.g., on-target/off-tumor effects or immunogenic effects), such as cytokine-driven toxicities (e.g., cytokine release syndrome (CRS)), infusion-related reactions (IRRs), macrophage activation syndrome (MAS), neurologic toxicities, severe tumor lysis syndrome (TLS), neutropenia, thrombocytopenia, elevated liver enzymes, and/or central nervous system (CNS) toxicities, following treatment with a HER2 TDB and an additional HER2 antibody (e.g., using the fractionated, dose-escalation dosing regimen of the invention) relative to treatment with the HER2 TDB in the absence of the additional HER2 antibody (e.g., with or without the use of a fractioned dosing regimen). In other embodiments, reduce or inhibit can refer to effector function of an antibody that is mediated by the antibody Fc region, such effector functions specifically including complement-dependent cytotoxicity (CDC), antibody-dependent cellular cytotoxicity (ADCC), and antibody-dependent cellular phagocytosis (ADCP).

“Tumor,” as used herein, refers to all neoplastic cell growth and proliferation, whether malignant or benign, and all pre-cancerous and cancerous cells and tissues. The terms “cancer,” “cancerous,” “cell proliferative disorder,” “proliferative disorder,” and “tumor” are not mutually exclusive as referred to herein.

As used herein, a “week” is 7 days±2 days.

As used herein, “administering” is meant a method of giving a dosage of a compound (e.g., a HER2 antibody or an additional HER2 antibody) or a composition (e.g., a pharmaceutical composition, e.g., a pharmaceutical composition including a HER2 antibody) to a subject. The compounds and/or compositions utilized in the methods described herein can be administered, for example, intravenously (e.g., by intravenous infusion), subcutaneously, intramuscularly, intradermally, percutaneously, intraarterially, intraperitoneally, intralesionally, intracranially, intraarticularly, intraprostatically, intrapleurally, intratracheally, intranasally, intravitreally, intravaginally, intrarectally, topically, intratumorally, peritoneally, subconjunctivally, intravesicularlly, mucosally, intrapericardially, intraumbilically, intraocularly, orally, topically, locally, by inhalation, by injection, by infusion, by continuous infusion, by localized perfusion bathing target cells directly, by catheter, by lavage, in cremes, or in lipid compositions. The method of administration can vary depending on various factors (e.g., the compound or composition being administered and the severity of the condition, disease, or disorder being treated). The term “package insert” is used to refer to instructions customarily included in commercial packages of therapeutic products, that contain information about the indications, usage, dosage, administration, combination therapy, contraindications and/or warnings concerning the use of such therapeutic products.

### II. Therapeutic Methods

The present invention provides improved methods of administering HER2 antibodies (e.g., a treatment regimen including administration of a HER2 TDB (e.g., BTRC4017A) and an additional HER2 antibody (e.g., a HER2 antibody that is not a TDB, such as trastuzumab)). Such methods can provide increased specificity to HER2-positive tumors, thereby reducing unwanted effects, such as on-target/off-tumor effects. The invention is based, in part, on the discovery that an increased therapeutic index can be achieved by co-treating a subject with a HER2 antibody (e.g., a bivalent, monospecific HER2 antibody, such as trastuzumab) and a HER2 TDB (e.g., BTRC4017) that binds the same HER2 domain as the HER2 antibody (e.g., domain IV of HER2). The increase in the therapeutic index can be associated with a decrease in likelihood of experiencing an on-target/off-tumor effect as compared to treatment with the HER2 TDB in the absence of the HER2 antibody. Additionally or alternatively, the increase in the therapeutic index can be associated with a decrease in likelihood of experiencing an immunogenic side effect as compared to treatment with the second HER2 antibody in the absence of the first HER2 antibody.

On-target/off-tumor effects can occur as a result of HER2 TDB binding to HER2 expressed by a non-tumor cell (e.g., a healthy cell). An on-target/off-tumor effect can be a symptom of pulmonary toxicity, such as interstitial lung disease, acute respiratory distress syndrome, dyspnea, cough, fatigue, or presence of pulmonary infiltrates. Additionally or alternatively, on-target/off-tumor effects may associated with dysfunction of healthy cells or tissues having a low or moderate expression of HER2, such as epithelial cells in the gastrointestinal tract, respiratory tract, reproductive tract, urinary tract, skin, breast, and placenta. Such on-target/off-tumor effects that can be reduced or inhibited by the methods described herein include, e.g., elevated liver enzyme levels, dry mouth, dry eyes, mucositis, esophagitis, or urinary symptoms.

In determining a therapeutic index, a dose sufficient to achieve a desired therapeutic effect can be determined according to a subject's objective response (OR) to the treatment regimen. In some embodiments, an OR is a complete response (CR) or a partial response (PR), according to RECIST v.1.1. Additionally or alternatively, a dose sufficient to achieve a desired therapeutic effect can be determined according to a subject's duration of response (DOR). In some embodiments, a DOR is the time from the first occurrence of a documented objective response to the time of the first documented disease progression or death from any cause, whichever occurs first, per RECIST v.1.1. Accordingly, in some embodiments, a method of the present invention increases a DOR and/or prolongs a subject's survival (e.g., overall survival (OS) or progression-free survival (PFS)).

In some embodiments, an increased therapeutic index resulting from a treatment regimen of the present invention is associated with a decreased likelihood of experiencing an immunogenic side effect as compared to treatment (e.g., an elevated level of anti-drug antibodies, an infusion/administration-related reaction (ARR), cardiac dysfunction, a pulmonary reaction, and cytokine release syndrome).

In determining a therapeutic index, a dose at which the therapeutic agent elicits a toxic effect can determined according to the presence of a dose-limiting toxicity (DLT), which is graded according to NCI CTCAE v5.0 (with the exception of cytokine release syndrome (CRS)). In some embodiments, a DLT is any of the following adverse events occurring during the assessment period (e.g., the first dosing cycle):

(a) ≥15% decrease from baseline in left ventricular ejection fraction (LVEF) or ≥10% decrease to less than a 50% LVEF;

(b) a hepatic function abnormality, e.g., as determined by the following:
(i) AST or ALT>3× the upper limit of normal (ULN) and total bilirubin >2×ULN, with the following exception: If the above occurs in the context of Grade ≤2 CRS (as defined by the criteria established by Lee et al. Blood 2014, 124:188-195), resolves to Grade s 1 within <3 days, and no individual laboratory value is Grade >3, this is not be considered a DLT;
(ii) any Grade 3 AST or ALT elevation with the following exception: If the above occurs in the context of Grade ≤2 CRS (as defined by the criteria established by Lee et al. Blood 2014, 124:188-195 resolves to Grade ≤1 within <3 days, this is not considered a DLT. In patients with metastatic liver lesions, Grade 3 transient increase of bilirubin, transaminases, and/or gamma-glutamyl transferase (GGT) that starts after infusion and recovers to Grade ≤2 or baseline within 1 week is not considered a DLT;

(c) Grade ≥3 lymphopenia lasting >7 days;

(d) Grade ≥4 neutropenia (ANC<500 cells/μL) lasting >7 days;

(e) Grade ≥3 febrile neutropenia;

(f) Grade ≥4 anemia;

(g) Grade ≥4 thrombocytopenia or Grade 3 thrombocytopenia associated with clinically significant bleeding; and/or

(h) Grade ≥3 non-hematologic, non-hepatic adverse event not attributable to another clearly identifiable cause, with the following exceptions: (i) Grade 3 nausea or vomiting that resolves to Grade ≤2 with standard-of-care therapy in ≤3 days; (ii) Grade 3 diarrhea, colitis, or enteritis that resolves to Grade ≤1 within 7 days with appropriate treatment; (iii) Grade 3 fatigue that resolves to Grade ≤2 in ≤7 days; (iv) Grade 3 fever (as defined by >40° C. for ≤24 hours); (v) Grade 3 laboratory abnormalities that are asymptomatic and considered by the investigator not to be clinically significant; (vi) Grade 3 rash that resolves to Grade ≤2 in ≤7 days with therapy equivalent to prednisone 10 mg/day or less; (vii) Grade 3 arthralgia that can be adequately managed with supportive care or that resolves to Grade ≤2 within 7 days; (viii) Grade 3 tumor flare defined as local pain, irritation, or rash localized at sites of known or suspected tumor that starts within 24 hours of infusion and resolves to Grade ≤2 in ≤7 days; (ix) Grade 3 hypoxia that starts within 24 hours of infusion and resolves to Grade ≤2 within 2 days after the start of the event; or (x) in patients with metastatic lung lesions, Grade 3 dyspnea secondary to localized lung edema that starts within 24 hours of infusion and recovers to Grade 1 or baseline within 2 days after the start of the event, and bronchospasm that resolves within 24 hours.

CRS is graded according to the Modified Cytokine Release Syndrome Grading System described in Russell et al. N. Engl. J. Med. 2008, 358:877-887 and Lee et al. Blood 2014, 124:188-195, and summarized in Tables 1 and 2, below.

TABLE 1
Modified Cytokine Release Syndrome Grading System

| Grade | Toxicity |
|---|---|
| Grade 1 | Symptoms are not life threatening and require symptomatic |
| | treatment only (e.g., fever, nausea, fatigue, headache, |
| | myalgias, malaise) |
| Grade 2 | Symptoms require and respond to moderate intervention |
| | Oxygen requirement <40%; or |
| | Hypotension responsive to fluids or low dose^{a} of one |
| | vasopressor; or Grade 2 organ toxicity |
| Grade 3 | Symptoms require and respond to aggressive intervention |
| | Oxygen requirement ≥40%; or |
| | Hypotension requiring high dose^{b} or multiple vasopressors; |
| | or Grade 3 organ toxicity or Grade 4 transaminitis |
| Grade 4 | Life-threatening symptoms |
| | Requirement for ventilator support or |
| | Grade 4 organ toxicity (excluding transaminitis) |
| Grade 5 | Death |

^{a}Low-dose vasopressor: single vasopressor at doses below that shown in Table 2 below.
^{b}High-dose vasopressor: as defined in Table 2 below.

TABLE 2
High-Dose Vasopressor (duration ≥3 hours)

| Pressor | Dose |
|---|---|
| Norepinephrine monotherapy | ≥20 mcg/min |
| Dopamine monotherapy | ≥10 mcg/kg/min |
| Phenylephrine monotherapy | ≥200 mcg/min |
| Epinephrine monotherapy | ≥10 mcg/min |
| If on vasopressin | Vasopressin + norepinephrine equivalent |
| | of ≥10 mcg/min^{a} |
| If on combination vasopressors | Norepinephrine equivalent of ≥20 |
| (not vasopressin) | mcg/min^{a} |

^{a}Norepinephrine equivalent dose = [norepinephrine (mcg/min)] + [dopamine (mcg/kg/min)] + [phenylephrine (mcg/min)/10]

In some instances, a treatment using the methods described herein by administering a HER2 TDB (e.g., a combination of a HER2 TDB with an additional HER2 antibody and/or a HER2 TDB in the context of a fractionated, dose-escalation dosing regimen) results in a reduction (e.g., by 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, 95% or greater, 96% or greater, 97% or greater, 98% or greater, or 99% or greater) or complete inhibition (100% reduction) of undesirable events, such as any one or more of the aforementioned events, following a treatment regimen of the invention relative to a control treatment regimen (e.g., a HER2 TDB monotherapy (in the absence of an additional HER2 antibody), or a HER2 TDB treatment in a non-fractioned dosing regimen).

In some instances, the increased therapeutic index resulting from a method of the invention is at least 1% increased relative to a control group (e.g., from 1% to 1,000% (10-fold) increased, from 2% to 5,000% increased, from 3% to 4,000% increased, from 4% to 3,000% increased, from 5% to 2,000% increased, from 10% to 1,000% increased, from 20% to 500% increased, or from 50% to 100% increased, e.g., from 1% to 5% increased, from 5% to 10% increased, from 10% to 20% increased, from 20% to 30% increased, from 30% to 40% increased, from 40% to 50% increased, from 50% to 60% increased, from 60% to 70% increased, from 70% to 80% increased, from 80% to 90% increased, from 90% to 100% increased, from 100% to 150% increased, from 150% to 200% increased, from 200% to 300% increased, from 300% to 400% increased, from 400% to 500% increased, or from 500% to 1,000% increased).

### HER2-Positive Cancers

The methods described herein can be used for treatment of HER2-positive cancers. In some instances, the HER2-positive cancer is a HER2-positive solid tumor. Additionally or alternatively, the HER2-positive cancer may be a locally advanced or metastatic HER2-positive cancer. In some instances, the HER2-positive cancer is a HER2-positive breast cancer or a HER2-positive gastric cancer. In some embodiments, the HER2-positive cancer is selected from the group consisting of a HER2-positive gastroesophageal junction cancer, a HER2-positive colorectal cancer, a HER2-positive lung cancer (e.g., a HER2-positive non-small cell lung carcinoma), a HER2-positive pancreatic cancer, a HER2-positive colorectal cancer, a HER2-positive bladder cancer, a HER2-positive salivary duct cancer, a HER2-positive ovarian cancer (e.g., a HER2-positive epithelial ovarian cancer), or a HER2-positive endometrial cancer.

Co-Treatment with a HER2 TDB and an Additional HER2 Antibody

The methods described herein include administering to a subject having cancer (e.g., a HER2-positive cancer) a HER2 TDB; e.g., a TDB that binds to HER2 and CD3, such as BTRC4017) and a HER2 antibody (e.g., an additional antibody that binds to HER2, e.g., a HER2 antibody that is not a HER2 TDB, such as a HER2 monospecific antibody (e.g., a monospecific, bivalent HER2 antibody)). In some embodiments, the HER2 TDB and the HER2 antibody both bind domain IV of HER2. For example, the HER2 TDB and the HER2 antibody may bind competitively to domain IV of HER2. In some embodiments, the HER2 TDB and the HER2 antibody bind HER2 at the same epitope or at an overlapping epitope (e.g., the same epitope or an overlapping epitope of HER2). In some embodiments, the HER2 TDB has a lower HER2 binding avidity than the additional HER2 antibody, which may be due, at least in part, to a lower HER2 valency of the HER2 TDB, relative to the additional antibody (e.g., wherein the HER2 TDB binds monovalently to HER2 and the additional HER2 antibody binds bivalently to HER2). Additionally or alternatively, a HER2 binding domain of the HER2 TDB may have the about same HER2 binding affinity as the additional HER2 antibody (e.g., the HER2 TDB and the additional HER2 antibody may share one, two, three, four, five, or all six CDRs; or one or both variable regions). In some embodiments, the HER2 TDB has a V_{H} and/or a V_{L} that shares at least 90%, at least 91%, at least 92%, at least 93%, at least 94%, at least 95%, at least 96%, at least 97%, at least 98%, at least 99%, or 100% amino acid sequence identity with a V_{H} and/or a V_{L} of the additional HER2 antibody. In some embodiments, the HER2 TDB and the HER2 antibody share the same HER2 binding domain (e.g., the HER2 binding domain of 4D5 (e.g., hu4D5), such as in the instance where the HER2 TDB is BTRC4017A and the HER2 antibody is trastuzumab, or an Fc modified variant thereof.

In some instances, any of the methods described herein may include administering a HER2 TDB that includes an anti-HER2 arm having a HER2 binding domain comprising at least one, two, three, four, five, or six complementarity-determining regions (CDRs) selected from (a) a CDR-H1 comprising the amino acid sequence of (SEQ ID NO: 1); (b) a CDR-H2 comprising the amino acid sequence of (SEQ ID NO: 2); (c) a CDR-H3 comprising the amino acid sequence of (SEQ ID NO:3); (d) a CDR-L1 comprising the amino acid sequence of (SEQ ID NO: 4); (e) a CDR-L2 comprising the amino acid sequence of (SEQ ID NO: 5); and (f) a CDR-L3 comprising the amino acid sequence of (SEQ ID NO: 6). In some instances, the HER2 TDB comprises an anti-HER2 arm comprising a HER2 binding domain comprising (a) a heavy chain variable domain (V_{H}) comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 7; (b) a light chain variable domain (V_{L}) comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 8; or (c) a V_{H} domain as in (a) and a V_{L} domain as in (b). Accordingly, in some instances, the HER2 binding domain comprises a V_{H} comprising an amino acid sequence of SEQ ID NO: 7 and a V_{L} comprising an amino acid sequence of SEQ ID NO: 8. An exemplary HER2 binding domain having the CDR and variable region sequences above is that of hu4D5, described, for example, in WO 2015/095392, which is incorporated herein by reference in its entirety.

In some instances, any of the methods described herein may include administering a HER2 TDB that includes an anti-CD3 arm having a CD3 binding domain comprising at least one, two, three, four, five, or six CDRs selected from (a) a CDR-H1 comprising the amino acid sequence of (SEQ ID NO: 9); (b) a CDR-H2 comprising the amino acid sequence of (SEQ ID NO: 10); (c) a CDR-H3 comprising the amino acid sequence of (SEQ ID NO: 11); (d) a CDR-L1 comprising the amino acid sequence of (SEQ ID NO: 12); (e) a CDR-L2 comprising the amino acid sequence of (SEQ ID NO: 13); and (f) a CDR-L3 comprising the amino acid sequence of (SEQ ID NO: 14). In some instances, the bispecific antibody comprises an anti-CD3 arm comprising a CD3 binding domain comprising (a) a V_{H} comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 15; (b) a V_{L} domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 16; or (c) a VH domain as in (a) and a VL domain as in (b). Accordingly, in some instances, the CD3 binding domain comprises a V_{H} domain comprising an amino acid sequence of SEQ ID NO: 15 and a V_{L} domain comprising an amino acid sequence of SEQ ID NO: 16. An exemplary CD3 binding domain having the CDR and variable region sequences above is that of 40G5c, described, for example, in WO 2015/095392, which is incorporated herein by reference in its entirety.

In some instances, any of the methods described herein may include administering a HER2 TDB that includes (i) an anti-HER2 arm having a HER2 binding domain comprising at least one, two, three, four, five, or six complementarity-determining regions (CDRs) selected from (a) a CDR-H1 comprising the amino acid sequence of (SEQ ID NO: 1); (b) a CDR-H2 comprising the amino acid sequence of (SEQ ID NO: 2); (c) a CDR-H3 comprising the amino acid sequence of (SEQ ID NO:3); (d) a CDR-L1 comprising the amino acid sequence of (SEQ ID NO: 4); (e) a CDR-L2 comprising the amino acid sequence of (SEQ ID NO: 5); and (f) a CDR-L3 comprising the amino acid sequence of (SEQ ID NO: 6); and (ii) an anti-CD3 arm having a CD3 binding domain comprising at least one, two, three, four, five, or six CDRs selected from (a) a CDR-H1 comprising the amino acid sequence of (SEQ ID NO: 9); (b) a CDR-H2 comprising the amino acid sequence of (SEQ ID NO: 10); (c) a CDR-H3 comprising the amino acid sequence of (SEQ ID NO: 11); (d) a CDR-L1 comprising the amino acid sequence of (SEQ ID NO: 12); (e) a CDR-L2 comprising the amino acid sequence of (SEQ ID NO: 13); and (f) a CDR-L3 comprising the amino acid sequence of (SEQ ID NO: 14). In some instances, the HER2 TDB comprises (i) an anti-HER2 arm comprising a HER2 binding domain comprising (a) a heavy chain variable domain (V_{H}) comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 7; (b) a light chain variable domain (V_{L}) comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 8; or (c) a V_{H} domain as in (a) and a V_{L} domain as in (b); and (ii) an anti-CD3 arm comprising a CD3 binding domain comprising (a) a V_{H} comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 15; (b) a V_{L} domain comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 16; or (c) a V_{H} domain as in (a) and a V_{L} domain as in (b). Accordingly, in some instances, the HER2 binding domain comprises a V_{H} comprising an amino acid sequence of SEQ ID NO: 7 and a V_{L} comprising an amino acid sequence of SEQ ID NO: 8, and the CD3 binding domain comprises a V_{H} domain comprising an amino acid sequence of SEQ ID NO: 15 and a V_{L} domain comprising an amino acid sequence of SEQ ID NO: 16. An exemplary such HER2 TDB is BTRC4017A, a full-length, “knob-in-hole” antibody that has a hu4D5 HER2 binding domain in an anti-HER2 arm paired with an anti-CD3 arm having a 40G5c CD3 binding domain.

In some instances, any of the methods described herein may include administering a HER2 TDB as described in WO 2015/063339. In some instances, any of the methods described herein may include administering the HER2 TDB GBR1302.

In some instances, any of the methods described herein may include administering a HER2 TDB having a monovalent arm and a bivalent arm. The monovalent arm may include a CD3 binding domain and the bivalent arm may include two HER2 binding domains, and each arm can have an Fc subunit that associates with the other Fc subunit (e.g., through a knob-in-hole configuration) to form an Fc domain. In this embodiment, the C-terminus of the CD3 binding domain is fused to the N-terminus of an Fc subunit, the C-terminus of one HER2 binding domain is fused to the N-terminus of a second HER2 binding domain, and the C-terminus of the second HER2 binding domain is fused to an N-terminus of the other Fc subunit. In some instances, the HER2 TDB having a monovalent arm and a bivalent arm binds domain IV of HER2. For example, the HER2 binding domains may have the hu4D5 sequence (e.g., trastuzumab) and/or the CD3 binding domain may have the 40G5c sequence. Examples of such bivalent HER2 TDBs are described in International Patent Application No. PCT/US2019/17251.

HER2 antibodies for co-treatment with a HER2 TDB include monospecific HER2 antibodies and multispecific (e.g., bispecific) HER2 antibodies (e.g., wherein the bispecific HER2 antibody is not a T cell-dependent bispecific antibody). In some embodiments, the HER2 antibody is multivalent (e.g., bivalent) to HER2. Additionally or alternatively, a HER2 antibody for use in the co-treatments described herein include full-length HER2-antibodies and HER2-binding fragments thereof. In instances involving full-length HER2-antibodies, an Fc region may include one or more modifications, e.g., to reduce effector function. Exemplary Fc modifications are discussed further in Section 5.c, below.

In some instances, any of the methods described herein may include administering a HER2 antibody (e.g., in addition to a HER2 TDB) that includes a HER2 binding domain comprising at least one, two, three, four, five, or six complementarity-determining regions (CDRs) selected from (a) a CDR-H1 comprising the amino acid sequence of (SEQ ID NO: 1); (b) a CDR-H2 comprising the amino acid sequence of (SEQ ID NO: 2); (c) a CDR-H3 comprising the amino acid sequence of (SEQ ID NO:3); (d) a CDR-L1 comprising the amino acid sequence of (SEQ ID NO: 4); (e) a CDR-L2 comprising the amino acid sequence of (SEQ ID NO: 5); and (f) a CDR-L3 comprising the amino acid sequence of (SEQ ID NO: 6). In some instances, the HER2 antibody comprises a HER2 binding domain comprising (a) a heavy chain variable domain (V_{H}) comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 7; (b) a light chain variable domain (V_{L}) comprising an amino acid sequence having at least 90% sequence identity (e.g., at least 91%, 92%, 93%, 94%, 95%, 96%, 97%, 98%, or 99% sequence identity) to, or the sequence of, SEQ ID NO: 8; or (c) a V_{H} domain as in (a) and a V_{L} domain as in (b). Accordingly, in some instances, the HER2 binding domain comprises a V_{H} comprising an amino acid sequence of SEQ ID NO: 7 and a V_{L} comprising an amino acid sequence of SEQ ID NO: 8. An exemplary HER2 binding domain having the CDR and variable region sequences above is that of hu4D5. In some embodiments, the HER2 antibody is trastuzumab. In other embodiments, the HER2 antibody is an Fc modified trastuzumab variant (e.g., trastuzumab-LALAPG).

The HER2 TDB and/or the additional HER2 antibody may be produced using recombinant methods and compositions, for example, as described in U.S. Pat. No. 4,816,567, which is incorporated herein by reference in its entirety.

In some instances, the HER2 TDB and/or the additional HER2 antibody according to any of the above embodiments described above may incorporate any of the features, singly or in combination, as described in Sections 1-5 below.

1. Antibody Affinity

In certain embodiments, the HER2 TDB and/or the additional HER2 antibody herein has a dissociation constant (K_{D}) of ≤1 μM, ≤100 nM, ≤10 nM, ≤1 nM, ≤0.1 nM, ≤0.01 nM, or ≤0.001 nM (e.g., 10⁻⁸ M or less, e.g., from 10⁻⁸ M to 10⁻¹³ M, e.g., from 10⁻⁹ M to 10⁻¹³ M) with respect to HER2 binding domain, the CD3 binding domain, or both.

In one embodiment, K_{D} is measured by a radiolabeled antigen binding assay (RIA). In one embodiment, an RIA is performed with the Fab version of an antibody of interest and its antigen. For example, solution binding affinity of Fabs for antigen is measured by equilibrating Fab with a minimal concentration of (¹²⁵I)-labeled antigen in the presence of a titration series of unlabeled antigen, then capturing bound antigen with an anti-Fab antibody-coated plate (see, e.g., Chen et al., J. Mol. Biol. 293:865-881(1999)). To establish conditions for the assay, MICROTITER® multi-well plates (Thermo Scientific) are coated overnight with 5 μg/ml of a capturing anti-Fab antibody (Cappel Labs) in 50 mM sodium carbonate (pH 9.6), and subsequently blocked with 2% (w/v) bovine serum albumin in PBS for two to five hours at room temperature (approximately 23° C.). In a non-adsorbent plate (Nunc #269620), 100 pM or 26 pM [¹²⁵I]-antigen are mixed with serial dilutions of a Fab of interest (e.g., consistent with assessment of the anti-VEGF antibody, Fab-12, in Presta et al., Cancer Res. 57:4593-4599 (1997)). The Fab of interest is then incubated overnight; however, the incubation may continue for a longer period (e.g., about 65 hours) to ensure that equilibrium is reached. Thereafter, the mixtures are transferred to the capture plate for incubation at room temperature (e.g., for one hour). The solution is then removed and the plate washed eight times with 0.1% polysorbate 20 (TWEEN-20®) in PBS. When the plates have dried, 150 μl/well of scintillant (MICROSCINT-20™; Packard) is added, and the plates are counted on a TOPCOUNT™ gamma counter (Packard) for ten minutes. Concentrations of each Fab that give less than or equal to 20% of maximal binding are chosen for use in competitive binding assays.

According to another embodiment, K_{D} is measured using a BIACORE® surface plasmon resonance assay. For example, an assay using a BIACORE®-2000 or a BIACORE®-3000 (BIAcore, Inc., Piscataway, N.J.) is performed at 25° C. with immobilized antigen CM5 chips at −10 response units (RU). In one embodiment, carboxymethylated dextran biosensor chips (CM5, BIACORE, Inc.) are activated with N-ethyl-N′-(3-dimethylaminopropyl)-carbodiimide hydrochloride (EDC) and N-hydroxysuccinimide (NHS) according to the supplier's instructions. Antigen is diluted with 10 mM sodium acetate, pH 4.8, to 5 μg/ml (˜0.2 μM) before injection at a flow rate of 5 μl/minute to achieve approximately 10 response units (RU) of coupled protein. Following the injection of antigen, 1 M ethanolamine is injected to block unreacted groups. For kinetics measurements, two-fold serial dilutions of Fab (0.78 nM to 500 nM) are injected in PBS with 0.05% polysorbate 20 (TWEEN-20™) surfactant (PBST) at 25° C. at a flow rate of approximately 25 μl/min. Association rates (kₒₙ) and dissociation rates (k_{off}) are calculated using a simple one-to-one Langmuir binding model (BIACORE® Evaluation Software version 3.2) by simultaneously fitting the association and dissociation sensorgrams. The equilibrium dissociation constant (K_{D}) is calculated as the ratio k_{off}/kₒₙ. See, for example, Chen et al., J. Mol. Biol. 293:865-881 (1999). If the on-rate exceeds 10M⁻¹s⁻¹ by the surface plasmon resonance assay above, then the on-rate can be determined by using a fluorescent quenching technique that measures the increase or decrease in fluorescence emission intensity (excitation=295 nm; emission=340 nm, 16 nm band-pass) at 25° C. of a 20 nM anti-antigen antibody (Fab form) in PBS, pH 7.2, in the presence of increasing concentrations of antigen as measured in a spectrometer, such as a stop-flow equipped spectrophometer (Aviv Instruments) or a 8000-series SLM-AMINCO™ spectrophotometer (ThermoSpectronic) with a stirred cuvette.

2. Antibody Fragments

In certain embodiments, the HER2 TDB and/or the additional HER2 antibody is an antibody fragment, e.g., an antibody fragment of a HER2 TDB binds to HER2 and CD3. Antibody fragments include, but are not limited to, Fab, Fab′, Fab′-SH, F(ab′)₂, Fv, and scFv fragments, and other fragments described below. For a review of certain antibody fragments, see Hudson et al. Nat. Med. 9:129-134 (2003). For a review of scFv fragments, see, e.g., Pluckthun, in The Pharmacology of Monoclonal Antibodies, vol. 113, Rosenburg and Moore eds., (Springer-Verlag, New York), pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046.

Diabodies are antibody fragments with two antigen-binding sites that may be bivalent or bispecific. See, for example, EP 404,097; WO 1993/01161; Hudson et al. Nat. Med. 9:129-134 (2003); and Hollinger et al. Proc. Natl. Acad. Sci. USA 90: 6444-6448 (1993). Triabodies and tetrabodies are also described in Hudson et al. Nat. Med. 9:129-134 (2003).

Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see, e.g., U.S. Pat. No. 6,248,516 B1).

Antibody fragments can be made by various techniques, including but not limited to proteolytic digestion of an intact antibody as well as production by recombinant host cells (e.g. E. coli or phage), as described herein.

3. Chimeric and Humanized Antibodies

In certain embodiments, the HER2 TDB and/or the additional HER2 antibody for use in accordance with the methods described herein is a chimeric antibody. Certain chimeric antibodies are described, e.g., in U.S. Pat. No. 4,816,567; and Morrison et al. Proc. Natl. Acad. Sci. USA, 81:6851-6855 (1984)). In one example, a chimeric antibody comprises a non-human variable region (e.g., a variable region derived from a mouse, rat, hamster, rabbit, or non-human primate, such as a monkey) and a human constant region. In a further example, a chimeric antibody is a “class switched” antibody in which the class or subclass has been changed from that of the parent antibody. Chimeric antibodies include antigen-binding fragments thereof.

In certain embodiments, a chimeric antibody is a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Humanized antibodies and methods of making them are reviewed, e.g., in Almagro and Fransson, Front. Biosci. 13:1619-1633 (2008), and are further described, e.g., in Riechmann et al., Nature 332:323-329 (1988); Queen et al., Proc. Nat'l Acad. Sci. USA 86:10029-10033 (1989); U.S. Pat. Nos. 5,821,337, 7,527,791, 6,982,321, and 7,087,409; Kashmiri et al., Methods 36:25-34 (2005) (describing specificity determining region (SDR) grafting); Padlan, Mol. Immunol. 28:489-498 (1991) (describing “resurfacing”); Dall'Acqua et al., Methods 36:43-60 (2005) (describing “FR shuffling”); and Osbourn et al., Methods 36:61-68 (2005) and Klimka et al., Br. J. Cancer, 83:252-260 (2000) (describing the “guided selection” approach to FR shuffling).

Human framework regions that may be used for humanization include but are not limited to: framework regions selected using the “best-fit” method (see, e.g., Sims et al. J. Immunol. 151:2296 (1993)); framework regions derived from the consensus sequence of human antibodies of a particular subgroup of light or heavy chain variable regions (see, e.g., Carter et al. Proc. Natl. Acad. Sci. USA, 89:4285 (1992); and Presta et al. J. Immunol., 151:2623 (1993)); human mature (somatically mutated) framework regions or human germline framework regions (see, e.g., Almagro and Fransson, Front. Biosci. 13:1619-1633 (2008)); and framework regions derived from screening FR libraries (see, e.g., Baca et al., J. Biol. Chem. 272:10678-10684 (1997) and Rosok et al., J. Biol. Chem. 271:22611-22618 (1996)).

4. Knob-In-Hole Bispecific Antibody Engineering

The HER2 TDB and/or the additional HER2 antibody may be prepared as a full-length antibody or an antibody fragment. Techniques for making bispecific antibodies include, but are not limited to, recombinant co-expression of two immunoglobulin heavy chain-light chain pairs having different specificities (see Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168). “Knob-in-hole” engineering of bispecific antibodies may be utilized to generate a first arm containing a knob and a second arm containing the hole into which the knob of the first arm may bind. The knob of the TDB may be on the anti-CD3 arm in one embodiment. Alternatively, the knob of the TDB of the invention may on the anti-HER2 arm. The hole of the TDB of the invention may be on the anti-CD3 arm in one embodiment. Alternatively, the hole of the TDB of the invention may be on the anti-HER2 arm. In some instances, the HER2 TDB and/or the additional HER2 antibody produced using knob-in-hole technology may comprise one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, second CH2 (CH2₂) domain, and a second CH3 (CH3₂) domain. In some instances, at least one of the one or more heavy chain constant domains is paired with another heavy chain constant domain. In some instances, the CH3₁ and CH3₂ domains each comprise a protuberance or cavity, and wherein the protuberance or cavity in the CH3₁ domain is positionable in the cavity or protuberance, respectively, in the CH3₂ domain. In some instances, the CH3₁ and CH3₂ domains meet at an interface between the protuberance and cavity. In some instances, the CH2₁ and CH2₂ domains each comprise a protuberance or cavity, and wherein the protuberance or cavity in the CH2₁ domain is positionable in the cavity or protuberance, respectively, in the CH2₂domain. In some instances, the CH2, and CH2₂ domains meet at an interface between said protuberance and cavity.

Bispecific antibodies (e.g., TDBs) may also be engineered using immunoglobulin crossover (also known as Fab domain exchange or CrossMab format) technology (see e.g., WO2009/080253; Schaefer et al., Proc. Natl. Acad. Sci. USA, 108:11187-11192 (2011)). Bispecific antibodies may also be made by engineering electrostatic steering effects for making antibody Fc-heterodimeric molecules (WO 2009/089004A1); cross-linking two or more antibodies or fragments (see, e.g., U.S. Pat. No. 4,676,980, and Brennan et al., Science, 229: 81 (1985)); using leucine zippers to produce bispecific antibodies (see, e.g., Kostelny et al. J. Immunol., 148(5):1547-1553 (1992)); using “diabody” technology for making bispecific antibody fragments (see, e.g., Hollinger et al., Proc. Nat. Acad. Sci. USA, 90:6444-6448 (1993)); and using single-chain Fv (sFv) dimers (see, e.g., Gruber et al., J. Immunol., 152:5368 (1994)); and preparing trispecific antibodies as described, e.g., in Tutt et al. J. Immunol. 147: 60 (1991).

The HER2 TDB and/or the additional HER2 antibody, or antibody fragments thereof, may also include a “Dual Acting FAb” or “DAF” comprising an antigen binding site that binds to a target other than HER2 (e.g., CD3, in the context of the HER2 TDB) as well as HER2 (see, e.g., U.S. Pub. No. 2008/0069820, which is incorporated herein by reference in its entirety).

5. Variants

In some instances, amino acid sequence variants of the HER2 TDB and/or the additional HER2 antibody described above are envisioned. For example, it may be desirable to improve the binding affinity and/or other biological properties of the one or both of the HER2 TDB and/or the additional HER2 antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, for example, antigen-binding.

a. Substitution, Insertion, and Deletion Variants

In certain embodiments, antibody variants having one or more amino acid substitutions are provided. Sites of interest for substitutional mutagenesis include the HVRs and FRs. Conservative substitutions are shown in Table 3 under the heading of “preferred substitutions.” More substantial changes are provided in Table 3 under the heading of “exemplary substitutions,” and as further described below in reference to amino acid side chain classes. Amino acid substitutions may be introduced into an antibody of interest and the products screened for a desired activity, for example, retained/improved antigen binding, decreased immunogenicity, or improved ADCC or CDC.

TABLE 3
Exemplary and Preferred Amino Acid Substitutions

| Original | Exemplary | Preferred |
|---|---|---|
| Residue | Substitutions | Substitutions |
| Ala (A) | Val; Leu; Ile | Val |
| Arg (R) | Lys; Gln; Asn | Lys |
| Asn (N) | Gln; His; Asp, Lys; Arg | Gln |
| Asp (D) | Glu; Asn | Glu |
| Cys (C) | Ser; Ala | Ser |
| Gln (Q) | Asn; Glu | Asn |
| Glu (E) | Asp; Gln | Asp |
| Gly (G) | Ala | Ala |
| His (H) | Asn; Gln; Lys; Arg | Arg |
| Ile (I) | Leu; Val; Met; Ala; Phe; Norleucine | Leu |
| Leu (L) | Norleucine; Ile; Val; Met; Ala; Phe | Ile |
| Lys (K) | Arg; Gln; Asn | Arg |
| Met (M) | Leu; Phe; Ile | Leu |
| Phe (F) | Trp; Leu; Val; Ile; Ala; Tyr | Tyr |
| Pro (P) | Ala | Ala |
| Ser (S) | Thr | Thr |
| Thr (T) | Val; Ser | Ser |
| Trp (W) | Tyr; Phe | Tyr |
| Tyr (Y) | Trp; Phe; Thr; Ser | Phe |
| Val (V) | Ile; Leu; Met; Phe; Ala; Norleucine | Leu |

Amino acids may be grouped according to common side-chain properties:
(1) hydrophobic: Norleucine, Met, Ala, Val, Leu, Ile;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Non-conservative substitutions will entail exchanging a member of one of these classes for another class.

One type of substitutional variant involves substituting one or more hypervariable region residues of a parent antibody (e.g. a humanized or human antibody). Generally, the resulting variant(s) selected for further study will have modifications (e.g., improvements) in certain biological properties (e.g., increased affinity, reduced immunogenicity) relative to the parent antibody and/or will have substantially retained certain biological properties of the parent antibody. An exemplary substitutional variant is an affinity matured antibody, which may be conveniently generated, e.g., using phage display-based affinity maturation techniques such as those described herein. Briefly, one or more CDR residues are mutated and the variant antibodies displayed on phage and screened for a particular biological activity (e.g. binding affinity).

Alterations (e.g., substitutions) may be made in CDRs, e.g., to improve antibody affinity. Such alterations may be made in CDR “hotspots,” i.e., residues encoded by codons that undergo mutation at high frequency during the somatic maturation process (see, e.g., Chowdhury, Methods Mol. Biol. 207:179-196 (2008)), and/or residues that contact antigen, with the resulting variant VH or VL being tested for binding affinity. Affinity maturation by constructing and reselecting from secondary libraries has been described, e.g., in Hoogenboom et al. in Methods in Molecular Biology 178:1-37 (O'Brien et al., ed., Human Press, Totowa, N.J., (2001).) In some embodiments of affinity maturation, diversity is introduced into the variable genes chosen for maturation by any of a variety of methods (e.g., error-prone PCR, chain shuffling, or oligonucleotide-directed mutagenesis). A secondary library is then created. The library is then screened to identify any antibody variants with the desired affinity. Another method to introduce diversity involves HVR-directed approaches, in which several HVR residues (e.g., 4-6 residues at a time) are randomized. HVR residues involved in antigen binding may be specifically identified, e.g., using alanine scanning mutagenesis or modeling. CDR-H3 and CDR-L3 in particular are often targeted.

In certain embodiments, substitutions, insertions, or deletions may occur within one or more CDRs so long as such alterations do not substantially reduce the ability of the antibody to bind antigen. For example, conservative alterations (e.g., conservative substitutions as provided herein) that do not substantially reduce binding affinity may be made in CDRs. Such alterations may, for example, be outside of antigen contacting residues in the CDRs. In certain embodiments of the variant VH and VL sequences provided above, each CDR either is unaltered, or contains no more than one, two or three amino acid substitutions.

A useful method for identification of residues or regions of an antibody that may be targeted for mutagenesis is called “alanine scanning mutagenesis” as described by Cunningham and Wells (1989) Science, 244:1081-1085. In this method, a residue or group of target residues (e.g., charged residues such as Arg, Asp, His, Lys, and Glu) are identified and replaced by a neutral or negatively charged amino acid (e.g., alanine or polyalanine) to determine whether the interaction of the antibody with antigen is affected. Further substitutions may be introduced at the amino acid locations demonstrating functional sensitivity to the initial substitutions. Alternatively, or additionally, a crystal structure of an antigen-antibody complex to identify contact points between the antibody and antigen. Such contact residues and neighboring residues may be targeted or eliminated as candidates for substitution. Variants may be screened to determine whether they contain the desired properties.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include an antibody with an N-terminal methionyl residue. Other insertional variants of the antibody molecule include the fusion to the N- or C-terminus of the antibody to an enzyme (e.g. for ADEPT) or a polypeptide which increases the serum half-life of the antibody.

b. Glycosylation Variants

In some instances, the methods of the invention involve administering to the subject a HER2 TDB and/or an additional HER2 antibody variant (e.g., in the context of a fractionated, dose-escalation dosing regimen) that has been modified to increase or decrease the extent to which the bispecific antibody is glycosylated. Addition or deletion of glycosylation sites to the HER2 TDB and/or the additional HER2 antibody of the invention may be conveniently accomplished by altering the amino acid sequence such that one or more glycosylation sites is created or removed.

Where the HER2 TDB and/or the additional HER2 antibody comprises an Fc region, the carbohydrate attached thereto may be altered. Native antibodies produced by mammalian cells typically comprise a branched, biantennary oligosaccharide that is generally attached by an N-linkage to Asn297 of the CH2 domain of the Fc region. See, e.g., Wright et al. TIBTECH 15:26-32 (1997). The oligosaccharide may include various carbohydrates, e.g., mannose, N-acetyl glucosamine (GlcNAc), galactose, and sialic acid, as well as a fucose attached to a GlcNAc in the “stem” of the biantennary oligosaccharide structure. In some embodiments, modifications of the oligosaccharide in an antibody of the invention may be made in order to create antibody variants with certain improved properties.

In some instances, the methods involve administering a HER2 TDB and/or additional HER2 antibody variant having a carbohydrate structure that lacks fucose attached (directly or indirectly) to an Fc region. For example, the amount of fucose in such antibody may be from 1% to 80%, from 1% to 65%, from 5% to 65% or from 20% to 40%. The amount of fucose is determined by calculating the average amount of fucose within the sugar chain at Asn297, relative to the sum of all glycostructures attached to Asn 297 (e. g. complex, hybrid and high mannose structures) as measured by MALDI-TOF mass spectrometry, as described in WO 2008/077546, for example. Asn297 refers to the asparagine residue located at about position 297 in the Fc region (EU numbering of Fc region residues); however, Asn297 may also be located about ±3 amino acids upstream or downstream of position 297, i.e., between positions 294 and 300, due to minor sequence variations in antibodies. Such fucosylation variants may have improved ADCC function. See, e.g., US Patent Publication Nos. US 2003/0157108 (Presta, L.); US 2004/0093621 (Kyowa Hakko Kogyo Co., Ltd). Examples of publications related to “defucosylated” or “fucose-deficient” antibody variants include: US 2003/0157108; WO 2000/61739; WO 2001/29246; US 2003/0115614; US 2002/0164328; US 2004/0093621; US 2004/0132140; US 2004/0110704; US 2004/0110282; US 2004/0109865; WO 2003/085119; WO 2003/084570; WO 2005/035586; WO 2005/035778; WO2005/053742; WO2002/031140; Okazaki et al. J. Mol. Biol. 336:1239-1249 (2004); Yamane-Ohnuki et al. Biotech. Bioeng. 87: 614 (2004). Examples of cell lines capable of producing defucosylated antibodies include Lec13 CHO cells deficient in protein fucosylation (Ripka et al. Arch. Biochem. Biophys. 249:533-545 (1986); US Pat Appl No US 2003/0157108 A1, Presta, L; and WO 2004/056312 A1, Adams et al., especially at Example 11), and knockout cell lines, such as alpha-1,6-fucosyltransferase gene, FUT8, knockout CHO cells (see, e.g., Yamane-Ohnuki et al. Biotech. Bioeng. 87: 614 (2004); Kanda, Y. et al., Biotechnol. Bioeng., 94(4):680-688 (2006); and WO2003/085107).

In view of the above, in some instances, the methods of the invention involve administering to the subject the HER2 TDB and/or the additional HER2 antibody variant (e.g., in the context of a fractionated, dose-escalation dosing regimen) that comprises an aglycosylation site mutation. In some instances, the aglycosylation site mutation reduces effector function of the HER2 TDB and/or the additional HER2 antibody. In some instances, the aglycosylation site mutation is a substitution mutation. In some instances, the bispecific antibody comprises a substitution mutation in the Fc region that reduces effector function. In some instances, the substitution mutation is at amino acid residue N297, L234, L235, and/or D265 (EU numbering). In some instances, the substitution mutation is selected from the group consisting of N297G, N297A, L234A, L235A, D265A, and P329G. In some instances, the substitution mutation is at amino acid residue N297. In a preferred embodiment, the substitution mutation is N297A.

In other instances, variants with bisected oligosaccharides are used in accordance with the methods of the invention, for example, in which a biantennary oligosaccharide attached to the Fc region of the antibody is bisected by GlcNAc. Such antibody variants may have reduced fucosylation and/or improved ADCC function. Examples of such antibody variants are described, e.g., in WO 2003/011878 (Jean-Mairet et al.); U.S. Pat. No. 6,602,684 (Umana et al.); and US 2005/0123546 (Umana et al.). Antibody variants with at least one galactose residue in the oligosaccharide attached to the Fc region are also provided. Such antibody variants may have improved CDC function. Such antibody variants are described, e.g., in WO 1997/30087 (Patel et al.); WO 1998/58964 (Raju, S.); and WO 1999/22764 (Raju, S.).

c. Fc Region Variants

In some instances, the HER2 TDB and/or the additional HER2 antibody variant that has one or more amino acid modifications introduced into the Fc region (i.e., an Fc region variant (see e.g., US 2012/0251531)) of the bispecific antibody may be administered to a subject having a HER2-positive cancer in accordance with the methods of the invention. The Fc region variant may comprise a human Fc region sequence (e.g., a human IgG1, IgG2, IgG3 or IgG4 Fc region) comprising an amino acid modification (e.g., a substitution) at one or more amino acid positions.

In some instances, the Fc region variant possesses some but not all effector functions, which makes it a desirable candidate for applications in which the half-life of the antibody in vivo is important yet certain effector functions (such as complement and ADCC) are unnecessary or deleterious. In vitro and/or in vivo cytotoxicity assays can be conducted to confirm the reduction/depletion of CDC and/or ADCC activities. For example, Fc receptor (FcR) binding assays can be conducted to ensure that the antibody lacks FcγR binding (hence likely lacking ADCC activity), but retains FcRn binding ability. The primary cells for mediating ADCC, NK cells, express Fc(RIII only, whereas monocytes express Fc(RI, Fc(RII and Fc(RIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest is described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assays methods may be employed (see, for example, ACTI™ non-radioactive cytotoxicity assay for flow cytometry (CellTechnology, Inc. Mountain View, Calif.; and CytoTox 96® non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in a animal model such as that disclosed in Clynes et al. Proc. Nat'l Acad. Sci. USA 95:652-656 (1998). C1q binding assays may also be carried out to confirm that the antibody is unable to bind C1 q and hence lacks CDC activity. See, e.g., C1q and C3c binding ELISA in WO 2006/029879 and WO 2005/100402. To assess complement activation, a CDC assay may be performed (see, for example, Gazzano-Santoro et al. J. Immunol. Methods 202:163 (1996); Cragg, M. S. et al. Blood. 101:1045-1052 (2003); and Cragg, M.S. and M.J. Glennie Blood. 103:2738-2743 (2004)). FcRn binding and in vivo clearance/half life determinations can also be performed using methods known in the art (see, e.g., Petkova, S. B. et al. Int'l. Immunol. 18(12):1759-1769 (2006)).

Antibodies with reduced effector function include those with substitution of one or more of Fc region residues 238, 265, 269, 270, 297, 327 and 329 (U.S. Pat. Nos. 6,737,056 and 8,219,149). Such Fc mutants include Fc mutants with substitutions at two or more of amino acid positions 265, 269, 270, 297 and 327, including the so-called “DANA” Fc mutant with substitution of residues 265 and 297 to alanine (U.S. Pat. Nos. 7,332,581 and 8,219,149).

In certain instances, the proline at position 329 of a wild-type human Fc region in the antibody is substituted with glycine or arginine or an amino acid residue large enough to destroy the proline sandwich within the Fc/Fcγ receptor interface that is formed between the proline 329 of the Fc and tryptophan residues Trp 87 and Trp 110 of FcgRIII (Sondermann et al. Nature. 406, 267-273 (2000)). In certain embodiments, the bispecific antibody comprises at least one further amino acid substitution. In one embodiment, the further amino acid substitution is S228P, E233P, L234A, L235A, L235E, N297A, N297D, or P331S, and still in another embodiment the at least one further amino acid substitution is L234A and L235A of the human IgG1 Fc region or S228P and L235E of the human IgG4 Fc region (see e.g., US 2012/0251531), and still in another embodiment the at least one further amino acid substitution is L234A and L235A and P329G of the human IgG1 Fc region.

Certain antibody variants with improved or diminished binding to FcRs are described. (See, e.g., U.S. Pat. No. 6,737,056; WO 2004/056312, and Shields et al., J. Biol. Chem. 9(2): 6591-6604 (2001).)

In certain instances, the HER2 TDB and/or the additional HER2 antibody comprises an Fc region with one or more amino acid substitutions which improve ADCC, e.g., substitutions at positions 298, 333, and/or 334 of the Fc region (EU numbering of residues).

In some instances, alterations are made in the Fc region that result in altered (i.e., either improved or diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

Antibodies with increased half-lives and improved binding to the neonatal Fc receptor (FcRn), which is responsible for the transfer of maternal IgGs to the fetus (Guyer et al., J. Immunol. 117:587 (1976) and Kim et al., J. Immunol. 24:249 (1994)), are described in US2005/0014934A1 (Hinton et al.). Those antibodies comprise an Fc region with one or more substitutions therein which improve binding of the Fc region to FcRn. Such Fc variants include those with substitutions at one or more of Fc region residues: 238, 256, 265, 272, 286, 303, 305, 307, 311, 312, 317, 340, 356, 360, 362, 376, 378, 380, 382, 413, 424 or 434, e.g., substitution of Fc region residue 434 (U.S. Pat. No. 7,371,826).

See also Duncan & Winter, Nature 322:738-40 (1988); U.S. Pat. Nos. 5,648,260; 5,624,821; and WO 94/29351 concerning other examples of Fc region variants.

d. Cysteine Engineered Antibody Variants

In certain embodiments, it may be desirable to create cysteine engineered HER2 TDBs and/or additional HER2 antibodies, e.g., “thioMAbs,” in which one or more residues of the bispecific antibody are substituted with cysteine residues. In particular embodiments, the substituted residues occur at accessible sites of the antibody. By substituting those residues with cysteine, reactive thiol groups are thereby positioned at accessible sites of the bispecific antibody and may be used to conjugate the antibody to other moieties, such as drug moieties or linker-drug moieties, to create an immunoconjugate. In certain embodiments, any one or more of the following residues may be substituted with cysteine: V205 (Kabat numbering) of the light chain; A118 (EU numbering) of the heavy chain; and S400 (EU numbering) of the heavy chain Fc region. Cysteine engineered antibodies may be generated as described, for example, in U.S. Pat. No. 7,521,541.

Therefore, immunoconjugates of an HER2 TDB and/or additional HER2 antibody conjugated to one or more cytotoxic agents, such as chemotherapeutic agents or drugs, growth inhibitory agents, toxins (e.g., protein toxins, enzymatically active toxins of bacterial, fungal, plant, or animal origin, or fragments thereof), or radioactive isotopes, are specifically contemplated.

In some instances, an immunoconjugate is an antibody-drug conjugate (ADC) in which an bispecific antibody is conjugated to one or more drugs, including but not limited to a maytansinoid (see U.S. Pat. Nos. 5,208,020, 5,416,064 and European Patent EP 0 425 235 B1); an auristatin such as monomethylauristatin drug moieties DE and DF (MMAE and MMAF) (see U.S. Pat. Nos. 5,635,483 and 5,780,588, and 7,498,298); a dolastatin; a calicheamicin or derivative thereof (see U.S. Pat. Nos. 5,712,374, 5,714,586, 5,739,116, 5,767,285, 5,770,701, 5,770,710, 5,773,001, and 5,877,296; Hinman et al., Cancer Res. 53:3336-3342 (1993); and Lode et al., Cancer Res. 58:2925-2928 (1998)); an anthracycline such as daunomycin or doxorubicin (see Kratz et al., Current Med. Chem. 13:477-523 (2006); Jeffrey et al., Bioorganic & Med. Chem. Letters 16:358-362 (2006); Torgov et al., Bioconj. Chem. 16:717-721 (2005); Nagy et al., Proc. Natl. Acad. Sci. USA 97:829-834 (2000); Dubowchik et al., Bioorg. & Med. Chem. Letters 12:1529-1532 (2002); King et al., J. Med. Chem. 45:4336-4343 (2002); and U.S. Pat. No. 6,630,579); methotrexate; vindesine; a taxane such as docetaxel, paclitaxel, larotaxel, tesetaxel, and ortataxel; a trichothecene; and CC1065.

In some instances, an immunoconjugate comprises the HER2 TDB and/or additional HER2 antibody conjugated to an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

In another embodiment, an immunoconjugate comprises the HER2 TDB and/or additional HER2 antibody conjugated to a radioactive atom to form a radioconjugate. A variety of radioactive isotopes are available for the production of radioconjugates. Examples include At²¹, I¹³¹, I¹²⁵, Y⁹⁰, Re¹⁸⁶, Re¹⁸⁸, Sm¹⁵³, Bi²¹², P³², Pb²¹² and radioactive isotopes of Lu. When the radioconjugate is used for detection, it may comprise a radioactive atom for scintigraphic studies, for example tc99m or 1123, or a spin label for nuclear magnetic resonance (NMR) imaging (also known as magnetic resonance imaging, mri), such as iodine-123 again, iodine-131, indium-111, fluorine-19, carbon-13, nitrogen-15, oxygen-17, gadolinium, manganese or iron.

Conjugates of the HER2 TDB and/or additional HER2 antibody and a cytotoxic agent may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). For example, a ricin immunotoxin can be prepared as described in Vitetta et al., Science 238:1098 (1987). Carbon-14-labeled 1-isothiocyanatobenzyl-3-methyldiethylene triaminepentaacetic acid (MX-DTPA) is an exemplary chelating agent for conjugation of radionucleotide to the antibody. See WO94/11026. The linker may be a “cleavable linker” facilitating release of a cytotoxic drug in the cell. For example, an acid-labile linker, peptidase-sensitive linker, photolabile linker, dimethyl linker or disulfide-containing linker (Chari et al., Cancer Res. 52:127-131 (1992); U.S. Pat. No. 5,208,020) may be used.

The immunuoconjugates or ADCs herein expressly contemplate, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate), which are commercially available (e.g., from Pierce Biotechnology, Inc., Rockford, Ill., U.S.A).

e. Other Antibody Derivatives

In some instances, the HER2 TDB and/or additional HER2 antibody may be modified to contain additional nonproteinaceous moieties that are known in the art and readily available and administered to the subject in accordance with the methods described herein. The moieties suitable for derivatization of the antibody include but are not limited to water soluble polymers. Non-limiting examples of water soluble polymers include, but are not limited to, polyethylene glycol (PEG), copolymers of ethylene glycol/propylene glycol, carboxymethylcellulose, dextran, polyvinyl alcohol, polyvinyl pyrrolidone, poly-1, 3-dioxolane, poly-1,3,6-trioxane, ethylene/maleic anhydride copolymer, polyaminoacids (either homopolymers or random copolymers), and dextran or poly(n-vinyl pyrrolidone)polyethylene glycol, propropylene glycol homopolymers, prolypropylene oxide/ethylene oxide co-polymers, polyoxyethylated polyols (e.g., glycerol), polyvinyl alcohol, and mixtures thereof. Polyethylene glycol propionaldehyde may have advantages in manufacturing due to its stability in water. The polymer may be of any molecular weight, and may be branched or unbranched. The number of polymers attached to the antibody may vary, and if more than one polymer are attached, they can be the same or different molecules. In general, the number and/or type of polymers used for derivatization can be determined based on considerations including, but not limited to, the particular properties or functions of the antibody to be improved, whether the antibody derivative will be used in a therapy under defined conditions, etc.

In some instances, conjugates of an antibody and nonproteinaceous moiety that may be selectively heated by exposure to radiation are provided. In one instance, the nonproteinaceous moiety is a carbon nanotube (Kam et al., Proc. Natl. Acad. Sci. USA 102: 11600-11605 (2005)). The radiation may be of any wavelength, and includes, but is not limited to, wavelengths that do not harm ordinary cells, but which heat the nonproteinaceous moiety to a temperature at which cells proximal to the antibody-nonproteinaceous moiety are killed.

### Dosing

HER2 TDBs and additional HER2 antibodies are dosed and administered in a fashion consistent with good medical practice. Treatment regimens provided herein include co-treatment of any of the HER2 TDBs described herein with an additional HER2 antibody (e.g., a HER2 antibody that is not a TDB, e.g., trastuzumab), wherein the additional HER2 antibody is administered prior to administration of the HER2 TDB (e.g., prior to the first administration of the HER2 TDB and/or prior to the administration of any subsequent administrations of the HER2 TDB).

In some embodiments, the HER2 antibody (e.g., a HER2 antibody that is not a TDB, e.g., trastuzumab) can be administered at a dose of about 5 mg/kg to about 10 mg/kg (e.g., 5 mg/kg to 10 mg/kg or 6 mg/kg to 8 mg/kg, e.g., about 5 mg/kg, about 6 mg/kg, about 7 mg/kg, about 8 mg/kg, about 9 mg/kg, or about 10 mg/kg). In some embodiments, the HER2 antibody (e.g., a HER2 antibody that is not a TDB, e.g., trastuzumab) is administered about once every three weeks (Q3W). The HER2 antibody can be infused (e.g., intravenously) over the course of at least about 30 minutes (e.g., 30-90 minutes). In some instances, e.g., at the first administration of the HER2 antibody, the HER2 antibody can be infused (e.g., intravenously) over the course of at least about 90 minutes and the subject may be observed for adverse reactions from the HER2 antibody over the course of 4-24 hours, e.g., prior to administration of a HER2 TDB. Alternatively, the HER2 antibody may be administered on the same day as the HER2 TDB (e.g., about 30-120 minutes after infusion of the HER2 antibody). In some embodiments, the duration between the first dose of a HER2 antibody and the first dose of the HER2 TDB is longer for the first dosing cycle than for subsequent dosing cycles. For example, the first dose of the HER2 antibody may be administered 24-hours prior to commencing the first dosing cycle by administering the first dose of the HER2 TDB, whereas subsequent dosing cycles include a HER2 antibody dose on the same day as the HER2 TDB dose (e.g., from 30 minutes to 120 minutes prior to the HER2 TDB dose).

In some instances, the HER2 TDB (e.g., BTRC4017A) is administered at a fixed dose. For example, a HER2 TDB can be administered at a fixed dose of 0.001 mg to 500 mg (e.g., from 0.003 mg to 250 mg, from 0.005 mg to 200 mg, from 0.01 mg to 150 mg, from 0.05 mg to 120 mg, from 0.1 mg to 100 mg, from 0.5 mg to 80 mg, or from 1.0 mg to 50 mg, e.g., from 0.001 mg to 0.005 mg, from 0.005 mg to 0.01 mg, from 0.01 mg to 0.05 mg, from 0.05 mg to 0.1 mg, from 0.1 mg to 0.5 mg, from 0.5 mg to 1.0 mg, from 1.0 mg to 5 mg, from 5 mg to 10 mg, from 10 mg to 20 mg, from 20 mg to 30 mg, from 30 mg to 40 mg, from 40 mg to 50 mg, from 50 mg to 60 mg, from 60 mg to 70 mg, from 70 mg to 80 mg, from 80 mg to 90 mg, from 90 mg to 100 mg, from 100 mg to 120 mg, from 120 mg to 150 mg, from 150 mg to 200 mg, from 200 mg to 250 mg, from 250 mg to 300 mg, from 300 mg to 350 mg, from 350 mg to 400 mg, from 400 mg to 450 mg, or from 450 mg to 500 mg, e.g., about 0.003 mg, about 0.005 mg, about 0.01 mg, about 0.05 mg, about 0.1 mg, about 0.5 mg, about 1.0 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, or about 250 mg, e.g., 0.003 mg, 0.009 mg, 0.027 mg, 0.081 mg, 0.24 mg, 0.72 mg, 1.08 mg, 1.51 mg, 2.2 mg, 2.3 mg, 4.0 mg, 4.6 mg, 6.6 mg, 8.0 mg, 9.2 mg, 12 mg, 13.2 mg, 14.8 mg, 18.4 mg, 19.8 mg, 26.4 mg, 36.8 mg, 51.5 mg, 52.8 mg, 61.3 mg, 72.1 mg, 105.6 mg, 147.8 mg, 176 mg, or 207 mg). In some embodiments, the HER2 TDB (e.g., BTRC4017A) is administered about once every three weeks (Q3W).

Methods provided herein include one-step fractionation treatment regimens. In a particular instance, a one-step fractionation treatment regimen includes a first dose of the HER2 antibody (e.g., trastuzumab) and, subsequently, a first dosing cycle (C1). The C1 includes a first dose of the HER2 TDB (e.g., BTRC2017A) (C1D1) and a second dose of the HER2 TDB (C1D2), wherein the C1D2 is greater than the C1D1 (e.g., at least two-fold the C1D1, e.g., from about two-fold to five-fold the C1D1, e.g., about two-fold or about three-fold the C1D1). A second dosing cycle (C2) is administered after the C1, wherein the C2 includes a second dose of the HER2 antibody (e.g., on day 1 of C1) and, subsequently (e.g., about 30-120 minutes after the second dose of the HER2 antibody), an additional dose of the HER2 TDB (C2D1). In such a one-step fractionation, the C2D1 can be equivalent to C1D2.

In some instances, the C1D1 is from 0.003 mg to 50 mg (e.g., from 0.003 mg to 50 mg, from 0.005 mg to 20 mg, from 0.01 mg to 10 mg, from 0.05 mg to 8 mg, or from 0.1 mg to 5 mg, e.g., from 0.001 mg to 0.005 mg, from 0.005 mg to 0.01 mg, from 0.01 mg to 0.05 mg, from 0.05 mg to 0.1 mg, from 0.1 mg to 0.5 mg, from 0.5 mg to 1.0 mg, from 1.0 mg to 5 mg, from 5 mg to 10 mg, from 10 mg to 20 mg, from 20 mg to 30 mg, from 30 mg to 40 mg, or from 40 mg to 50 mg, e.g., about 0.003 mg, about 0.005 mg, about 0.01 mg, about 0.05 mg, about 0.1 mg, about 0.5 mg, about 1.0 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 30 mg, about 35 mg, about 40 mg, about 45 mg, or about 50 mg). In some embodiments, the C1D1 is 0.003 mg, 0.009 mg, 0.027 mg, 0.081 mg, 0.12 mg, 0.24 mg, 0.48 mg, 0.72 mg, 1.0 mg, 2.0 mg, 2.2 mg, 4.0 mg, 6.6 mg, 8.0 mg, 12 mg, 18 mg, 27 mg, or 40.5 mg. The C1D2 can be from 0.009 mg to 200 mg (e.g., from 0.01 mg to 150 mg, from 0.05 mg to 100 mg, from 0.1 mg to 50 mg, from 0.5 mg to 20 mg, or from 1 mg to 10 mg, e.g., from 0.009 mg to 0.01 mg, from 0.01 mg to 0.05 mg, from 0.05 mg to 0.1 mg, from 0.1 mg to 0.5 mg, from 0.5 mg to 1.0 mg, from 1.0 mg to 5 mg, from 5 mg to 10 mg, from 10 mg to 20 mg, from 20 mg to 30 mg, from 30 mg to 40 mg, from 40 mg to 50 mg, from 50 mg to 60 mg, from 60 mg to 70 mg, from 70 mg to 80 mg, from 80 mg to 90 mg, from 90 mg to 100 mg, from 100 mg to 120 mg, from 120 mg to 150 mg, or from 150 mg to 200 mg, e.g., about 0.009 mg, about 0.01 mg, about 0.05 mg, about 0.1 mg, about 0.5 mg, about 1.0 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, or about 200 mg). In some embodiments, the C1D2 is 0.009 mg, 0.027 mg, 0.081 mg, 0.24 mg, 0.4 mg, 0.72 mg, 0.08 mg, 1.6 mg, 2.2 mg, 2.3 mg, 3.2 mg, 4.6 mg, 6.4 mg, 6.6 mg, 9.2 mg, 12.8 mg, 14.8 mg, 18.4 mg, 19.8 mg, 25.6 mg, 36.8 mg, 38.4, 51.5 mg, 57.6 mg, 72.1 mg, 86.4 mg, 61.3 mg, or 129.6 mg.

In a one-step fractionation treatment regimen, the C1D1 and C1D2 are administered on different days within the C1. In some embodiments, e.g., in which the C1 is 21 days, the C1D1 is administered on C1 day 1, and the C1D2 is administered on C1 day 8.

Further provided herein are two-step fractionation treatment regimens, which include a third HER2 TDB dose in the first dosing cycle (C1D3). C1D3 is greater than C1D2, which is greater than C1D1. In some embodiments, the C1D1, the C1D2, and the C1D3 are cumulatively greater than a highest cleared dose of the HER2 TDB in a first dosing cycle of a one-step fractionation, dose-escalation dosing regimen. For example, in a study using a one-step fractionation, dose-escalation dosing regimen, in which the highest cleared dose is determined to be 20 mg in C1, the sum of C1D1, C1D2, and C1D3 in a two-step fractionation treatment regimen can be greater than 20 mg (e.g., about 25 mg). In such two-step fractionation treatment regimens, the C1D2 can two-fold to ten-fold (e.g., about two-fold, about three-fold, about four-fold, about five-fold, about six-fold, about seven-fold, about eight-fold, about nine-fold, or about ten-fold) the dose of the C1D1. Additionally or alternatively, the C1D3 can be from two-fold to three-fold the dose of the C1D2. In some instances, the C2D1 is equivalent to the C1D3.

In particular instances of a two-step fractionation treatment regimen, the C1D1 can be from 0.01 mg to 20 mg (e.g., from 0.05 mg to 15 mg, from 0.1 mg to 10 mg, or from 0.5 mg to 5 mg, e.g., from 0.01 mg to 0.05 mg, from 0.05 mg to 0.1 mg, from 0.1 mg to 0.5 mg, from 0.5 mg to 1.0 mg, from 1.0 mg to 5 mg, from 5 mg to 10 mg, from 10 mg to 15 mg, or from 15 mg to 20 mg, e.g., about 0.01 mg, about 0.05 mg, about 0.1 mg, about 0.5 mg, about 1.0 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, or about 20 mg). Additionally or alternatively, the C1D2 can be from 0.1 mg to 100 mg (e.g., from 0.1 mg to 80 mg, from 0.5 mg to 50 mg, or from 1 mg to 10 mg, e.g., from 0.1 mg to 0.5 mg, from 0.5 mg to 1.0 mg, from 1.0 mg to 5 mg, from 5 mg to 10 mg, from 10 mg to 20 mg, from 20 mg to 30 mg, from 30 mg to 40 mg, from 40 mg to 50 mg, from 50 mg to 60 mg, from 60 mg to 70 mg, from 70 mg to 80 mg, from 80 mg to 90 mg, or from 90 mg to 100 mg, e.g., about 0.01 mg, about 0.05 mg, about 0.1 mg, about 0.5 mg, about 1.0 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, or about 100 mg). Thus, the C1D3 can range from 1 mg to 400 mg (e.g., from 10 mg to 300 mg, from 20 mg to 200 mg, or from 50 mg to 100 mg, e.g., from 1.0 mg to 5 mg, from 5 mg to 10 mg, from 10 mg to 20 mg, from 20 mg to 30 mg, from 30 mg to 40 mg, from 40 mg to 50 mg, from 50 mg to 60 mg, from 60 mg to 70 mg, from 70 mg to 80 mg, from 80 mg to 90 mg, from 90 mg to 100 mg, from 100 mg to 120 mg, from 120 mg to 150 mg, from 150 mg to 200 mg, from 200 to 250 mg, from 250 mg to 300 mg, from 300 mg to 350 mg, or from 350 mg to 400 mg, e.g., about 1.0 mg, about 2 mg, about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg, about 16 mg, about 17 mg, about 18 mg, about 19 mg, about 20 mg, about 21 mg, about 22 mg, about 23 mg, about 24 mg, about 25 mg, about 30 mg, about 35 mg, about 40 mg, about 45 mg, about 50 mg, about 55 mg, about 60 mg, about 65 mg, about 70 mg, about 75 mg, about 80 mg, about 85 mg, about 90 mg, about 95 mg, about 100 mg, about 150 mg, about 200 mg, about 250 mg, about 300 mg, about 350 mg, or about 400 mg). In some embodiments, the C1D3 is 1.1 mg, 2.2 mg, 4.4 mg, 6.6 mg, 8.8 mg, 13.2 mg, 17.6 mg, 26.4 mg, 35.2 mg, 52.8 mg, 70.4 mg, 105.6 mg, 147.8 mg, 158.4 mg, 176 mg, 207 mg, 237.6 mg, or 356.4 mg.

In a two-step fractionation treatment regimen, the C1D1, C1D2, and C1D3 are administered on different days within the C1. In some embodiments, e.g., in which the C1 is 21 days, the C1D1 is administered on C1 day 1, the C1D2 is administered on C1 day 8, and the C1D3 is administered on day 15.

In some instances of any of the aforementioned treatment regimens, the second and any subsequent dosing cycles are the same duration as the first dosing cycle (e.g., 7-42 days, 14-35 days, or 21-28 days, e.g., 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42 days, or longer). In some embodiments, the C1, C2, C3, and all subsequent cycles (e.g., C4, C5, C6, etc.) are each about 21 days. The HER2 TDB and the HER2 antibody may both be administered on day 1 of each cycle after C1.

The duration of therapy will continue for as long as medically indicated or until a desired therapeutic effect (e.g., those described herein) is achieved. In certain embodiments, the treatment is continued for 1 month, 2 months, 4 months, 6 months, 8 months, 10 months, 1 year, 2 years, 3 years, 4 years, 5 years, or for a period of years up to the lifetime of the subject.

For all the methods described herein, the one or more HER2 antibodies are formulated, dosed, and administered in a fashion consistent with good medical practice. Factors for consideration in this context include the particular disorder being treated, the particular mammal being treated, the clinical condition of the individual patient, the cause of the disorder, the site of delivery of the agent, the method of administration, the scheduling of administration, and other factors known to medical practitioners. The one or more HER2 antibodies need not be, but is optionally formulated with, one or more agents currently used to prevent or treat the disorder in question. The effective amount of such other agents depends on the amount of the HER2 antibody present in the formulation, the type of disorder or treatment, and other factors discussed above. The one or more HER2 antibodies may be suitably administered to the patient over a series of treatments.

### Additional Therapeutic Agents

In some instances of any of the presently described methods of treating subject having a HER2-positive cancer, a treatment regimen may include administration of one or more additional therapeutic agents.

In one instance, the additional therapeutic agent is a corticosteroid, which can be administered as a pre-treatment prior to (e.g., about 1 hour prior to) administration of a HER2 TDB or an additional HER2 antibody. Corticosteroid premedication can include administration of dexamethasone or methylprednisolone. Additionally or alternatively, the treatment regimens described herein may include administration of (e.g., pre-treatment with) acetaminophen, paracetamol, or diphenhydramine.

In some instances, an IL-6R antagonist, such as tocilizumab (ACTEMRA®/RoACTEMRA®) is administered, e.g., if necessary to manage a CRS event. In particular embodiments, an IL-6R antagonist (e.g., tocilizumab) is administered intravenously, e.g., at a dose of 1 mg/kg to 25 mg/kg (e.g., from 5 mg/kg to 10 mg/kg, e.g., about 8 mg/kg), as necessary.

In some instances, any of the treatment regimens described herein include administration of a PD-1 axis binding antagonist (e.g., a PD-L1 binding antagonist, a PD-1 binding antagonist, or a PD-L2 binding antagonist).

In some instances, a PD-L1 binding antagonist is an anti-PD-L1 antibody selected from MPDL3280A (atezolizumab), YW243.55.S70, MDX-1105, and MEDI4736 (durvalumab), and MSB0010718C (avelumab). Antibody YW243.55.S70 is an anti-PD-L1 described in PCT Pub. No. WO 2010/077634. MDX-1105, also known as BMS-936559, is an anti-PD-L1 antibody described in PCT Pub. No. WO 2007/005874. MEDI4736 (durvalumab) is an anti-PD-L1 monoclonal antibody described in PCT Pub. No. WO 2011/066389 and U.S. Pub. No. 2013/034559. Examples of anti-PD-L1 antibodies useful for the methods of this invention, and methods for making thereof are described in PCT Pub. Nos. WO 2010/077634, WO 2007/005874, and WO 2011/066389, and also in U.S. Pat. No. 8,217,149, and U.S. Pub. No. 2013/034559, which are incorporated herein by reference.

In some instances, the PD-1 binding antagonist is another anti-PD-1 antibody, such as an anti-PD-1 antibody selected from the group consisting of MDX-1106 (nivolumab), MK-3475 (pembrolizumab), MEDI-0680 (AMP-514), PDR001, REGN2810, and BGB-108. MDX-1106, also known as MDX-1106-04, ONO-4538, BMS-936558, or nivolumab, is an anti-PD-1 antibody described in PCT Pub. No. WO 2006/121168. MK-3475, also known as pembrolizumab or lambrolizumab, is an anti-PD-1 antibody described in PCT Pub. No. WO 2009/114335. In other instances, the PD-1 binding antagonist is an immunoadhesin (e.g., an immunoadhesin comprising an extracellular or PD-1 binding portion of PD-L1 or PD-L2 fused to a constant region (e.g., an Fc region of an immunoglobulin sequence). In other instances, the PD-1 binding antagonist is AMP-224. AMP-224, also known as B7-DCIg, is a PD-L2-Fc fusion soluble receptor described in PCT Pub. Nos. WO 2010/027827 and WO 2011/066342.

In other instances, the PD-L2 binding antagonist is an anti-PD-L2 antibody (e.g., a human, a humanized, or a chimeric anti-PD-L2 antibody). In some instances, the PD-L2 binding antagonist is an immunoadhesin.

In a further embodiment, an additional therapeutic agent is a further chemotherapy agent and/or an antibody-drug conjugate (ADC). In one embodiment, the HER2 TDB and/or HER2 antibody is co-administered with one or more additional chemotherapy agents selected from cyclophosphamide, doxorubicin, vincristine, and prednisolone (CHOP). In one embodiment, a HER2 TDB and/or HER2 antibody is co-administered with an ADC selected from an anti-CD79b antibody drug conjugate (such as anti-CD79b-MC-vc-PAB-MMAE or the anti-CD79b antibody drug conjugate described in any one of U.S. Pat. No. 8,088,378 and/or US 2014/0030280, or polatuzumab vedotin).

In some instances, the additional therapy includes an alkylating agent. In one instance, the alkylating agent is 4-[5-[bis(2-chloroethyl)amino]-1-methylbenzimidazol-2-yl]butanoic acid and salts thereof. In one instance, the alkylating agent is bendamustine.

In some instances, the additional therapy comprises a BCL-2 inhibitor. In one embodiment, the BCL-2 inhibitor is 4-(4-{[2-(4-chlorophenyl)-4,4-dimethylcyclohex-1-en-1-yl]methyl}piperazin-1-yl)-N-({3-nitro-4-[(tetrahydro-2H-pyran-4-ylmethyl)amino]phenyl}sulfonyl)-2-(1H-pyrrolo[2,3-b]pyridin-5-yloxy)benzamide and salts thereof. In one instance, the BCL-2 inhibitor is venetoclax (CAS #: 1257044-40-8).

In some instances, the additional therapy comprises a phosphoinositide 3-kinase (PI3K) inhibitor. In one instance, the PI3K inhibitor inhibits delta isoform PI3K (i.e., P110δ). In some instances, the PI3K inhibitor is 5-Fluoro-3-phenyl-2-[(1 S)-1-(7H-purin-6-ylamino)propyl]-4(3H)-quinazolinone and salts thereof. In some instances, the PI3K inhibitor is idelalisib (CAS #: 870281-82-6). In one instance, the PI3K inhibitor inhibits alpha and delta isoforms of PI3K. In some instances, the PI3K inhibitor is 2-{3-[2-(1-Isopropyl-3-methyl-1H-1,2-4-triazol-5-yl)-5,6-dihydrobenzo[f]imidazo[1,2-d][1,4]oxazepin-9-yl]-1H-pyrazol-1-yl}-2-methylpropanamide and salts thereof.

In a further aspect of the invention, the additional therapy comprises a Bruton's tyrosine kinase (BTK) inhibitor. In one instance, the BTK inhibitor is 1-[(3R)-3-[4-Amino-3-(4-phenoxyphenyl)-1H-pyrazolo[3,4-d]pyrimidin-1-yl]piperidin-1-yl]prop-2-en-1-one and salts thereof. In one instance, the BTK inhibitor is ibrutinib (CAS #: 936563-96-1).

In some instances, the additional therapy comprises thalidomide or a derivative thereof. In one instance, the thalidomide or a derivative thereof is (RS)-3-(4-Amino-1-oxo-1,3-dihydro-2H-isoindol-2-yl)piperidine-2,6-dione and salts thereof. In one instance, the thalidomide or a derivative thereof is lendalidomide (CAS #: 191732-72-6).

### Pharmaceutical Compositions and Formulations

Pharmaceutical compositions and formulations of the HER2 TDBs and/or HER2 antibodies described above can be prepared by mixing such agents having the desired degree of purity with one or more optional pharmaceutically acceptable carriers (Remington's Pharmaceutical Sciences 16th edition, Osol, A. Ed. (1980)), in the form of lyophilized formulations or aqueous solutions. Pharmaceutically acceptable carriers are generally nontoxic to recipients at the dosages and concentrations employed, and include, but are not limited to: buffers such as phosphate, citrate, and other organic acids; antioxidants including ascorbic acid and methionine; preservatives (such as octadecyldimethylbenzyl ammonium chloride; hexamethonium chloride; benzalkonium chloride; benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes (e.g. Zn-protein complexes); and/or non-ionic surfactants such as polyethylene glycol (PEG). Exemplary pharmaceutically acceptable carriers herein further include insterstitial drug dispersion agents such as soluble neutral-active hyaluronidase glycoproteins (sHASEGP), for example, human soluble PH-20 hyaluronidase glycoproteins, such as rHuPH20 (HYLENEX®, Baxter International, Inc.). Certain exemplary sHASEGPs and methods of use, including rHuPH20, are described in U.S. Patent Publication Nos. 2005/0260186 and 2006/0104968. In one aspect, a sHASEGP is combined with one or more additional glycosaminoglycanases such as chondroitinases.

Exemplary lyophilized antibody formulations are described in U.S. Pat. No. 6,267,958. Aqueous antibody formulations include those described in U.S. Pat. No. 6,171,586 and WO2006/044908, the latter formulations including a histidine-acetate buffer.

The formulation herein may also contain more than one active ingredients as necessary for the particular indication being treated, preferably those with complementary activities that do not adversely affect each other. For example, it may be desirable to further provide an additional therapeutic agent (e.g., a chemotherapeutic agent, a cytotoxic agent, a growth inhibitory agent, and/or an anti-hormonal agent, such as those recited herein above). Such active ingredients are suitably present in combination in amounts that are effective for the purpose intended.

Active ingredients may be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington's Pharmaceutical Sciences 16th edition, Osol, A. Ed. (1980).

Sustained-release preparations may be prepared. Suitable examples of sustained-release preparations include semipermeable matrices of solid hydrophobic polymers containing the antibody, which matrices are in the form of shaped articles, for example, films, or microcapsules.

The formulations to be used for in vivo administration are generally sterile. Sterility may be readily accomplished, e.g., by filtration through sterile filtration membranes.

### III. Articles of Manufacture

The invention further provides articles of manufacture containing materials useful for the treatment or prevention of a HER2-positive cancer. The article of manufacture comprises a container and a label or package insert on or associated with the container. Suitable containers include, for example, bottles, vials, syringes, IV solution bags, etc. The containers may be formed from a variety of materials such as glass or plastic. The container holds a composition which is by itself or combined with another composition effective for treating or preventing the condition and may have a sterile access port (for example the container may be an intravenous solution bag or a vial having a stopper pierceable by a hypodermic injection needle). At least one active agent in the composition is a HER TDB described herein. The label or package insert indicates that the composition is used for treating the HER2-positive cancer of choice (e.g., a HER2-positive breast cancer or a HER2-positive gastric cancer) and further includes information related to at least one of the dosing regimens described herein. Moreover, the article of manufacture may include (a) a first container with a composition contained therein, wherein the composition comprises a HER2 TDB; and (b) a second container with a composition contained therein, wherein the composition comprises a further HER antibody (e.g., a multivalent (e.g., bivalent) HER2-binding antibody, e.g., trastuzumab). Alternatively, or additionally, the article of manufacture may further include one or more additional containers containing (a) an additional therapeutic agent; and/or (b) a pharmaceutically-acceptable buffer, such as bacteriostatic water for injection (BWFI), phosphate-buffered saline, Ringer's solution and dextrose solution. Containers may further include other materials desirable from a commercial and user standpoint, including other buffers, diluents, filters, needles, and syringes.

### IV. Examples

The following are examples of the methods of the invention. It is understood that various other embodiments may be practiced, given the general description provided above.

### Example 1. Preclinical Efficacy of BTRC4017A and Trastuzumab Co-Treatment

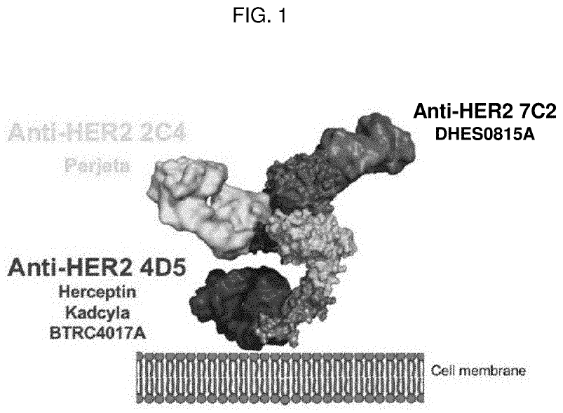

A full-length, IgG₁ TDB, BTRC4017A, that binds both HER2 and CD3 was generated using “knob-in-hole” engineering (see, e.g., U.S. Pat. No. 5,731,168), and has an anti-HER2 arm including a 4D5 HER2 binding site and an anti-CD3 arm including a 40G5c CD3 binding site (see, e.g., WO 2015/095392). The 4D5 HER2-binding site of BTRC4017A is derived from trastuzumab (HERCEPTIN®) and binds the same epitope in domain IV of HER2, as illustrated in FIG. 1. Trastuzumab competes with BTRC4017A for binding to HER2 and can therefore interfere with BTRC4017A activity.

In Vitro Pharmacology of BTRC4017A in Combination with Trastuzumab (Herceptin)

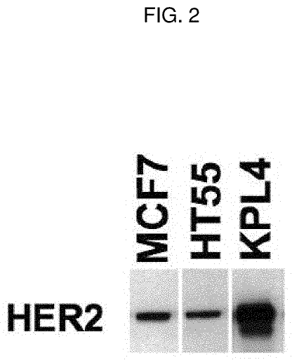

The impact of trastuzumab on BTRC4017A activity was tested in vitro and in vivo using HER2-amplified KPL4 cell line, which represents a HER2-positive cancer. The impact of the combination was also modeled using an HT55 cell line, which expresses low levels of HER2, similar to normal human tissue HER2 levels. HT55 tumors were used to model on-target activity on normal cells/tissues that express low levels of HER2, whereas KPL4 tumor cells represent a HER2-overexpressing tumor. MCF7 is a HER2 IHC0 breast cancer cell-line, included as a negative control. HER2 expression levels in the model cell lines are shown in FIG. 2.

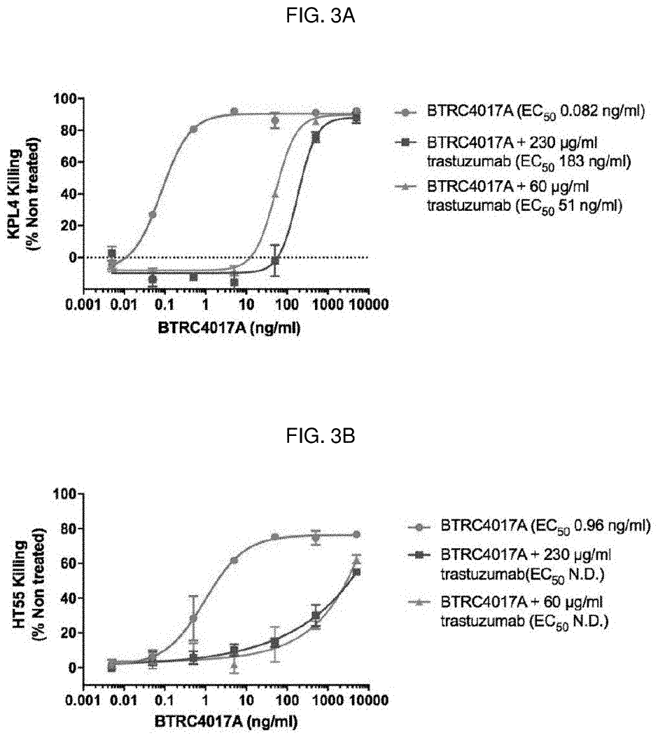

To evaluate the effect of trastuzumab on the in vitro activity of BTRC4017A, dose-response experiments were performed in the presence of 230 μg/mL and 60 μg/mL trastuzumab. These trastuzumab concentrations represent clinical maximum serum concentration (Cₘₐₓ) and minimum serum concentration (Cₘᵢₙ) in breast cancer at recommended doses (8 or 6 mg/kg every 3 weeks [Q3W]). Purified human CD8⁺ T cells were used as effectors to minimize target cell killing by trastuzumab via cell-mediated ADCC. Trastuzumab inhibited the activity of BTRC4017A in both cell lines (FIGS. 3A and 3B), and 600-fold to 2000-fold more BTRC4017A was required to reach the 50% effective concentration of KPL4 (FIG. 3A) killing in presence of trastuzumab. The inhibitory effect of trastuzumab on BTRC4017A activity was in a similar range when HT55 cells were targeted, but at high concentrations, BTRC4017A was able to overcome the inhibitory effect of trastuzumab in both cell lines.

In Vivo Pharmacology of BTRC4017A in Combination with Trastuzumab-LALAPG

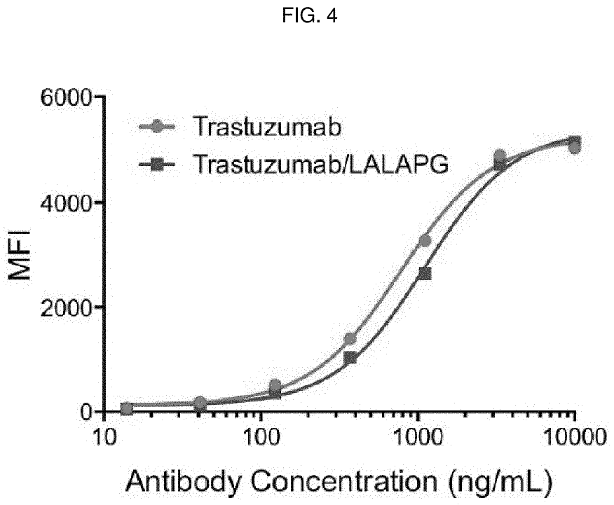

Fc receptor-mediated effector functions play a major role in the in vivo activity of trastuzumab and can interfere with in vivo experiments addressing inhibitory effect of trastuzumab on BTRC4017A activity. The Fc-region of trastuzumab was therefore modified by introducing a set of amino acid substitutions that attenuate effector functions of human IgG₁ to generate a trastuzumab-LALAPG variant. The LALAPG mutations are L234A, L235A, and P329G. As the anti-HER2 Fab in trastuzumab was not modified in the trastuzumab-LALAPG variant, the modification did not alter HER2 binding, as confirmed with a flow cytometry HER2 binding assay (FIG. 4).

To test the effect of trastuzumab/LALAPG on BTRC4017A activity, a dual tumor mouse model based on highly immune compromised NOD scid gamma (NSG) mice was used. NSG mice were supplemented with human T cells by intraperitoneal injection of human peripheral mononuclear cells (PBMC). In each mouse, a KPL4 tumor was grafted on one flank, and a HT55 tumor was grafted on the opposite flank. KPL4 is a HER2-amplified cell line and represents a HER2-overexpressing tumor, whereas HT55 tumors were used to model on-target/off-tumor activity on normal cells/tissues that express low levels of HER2 (FIG. 2).

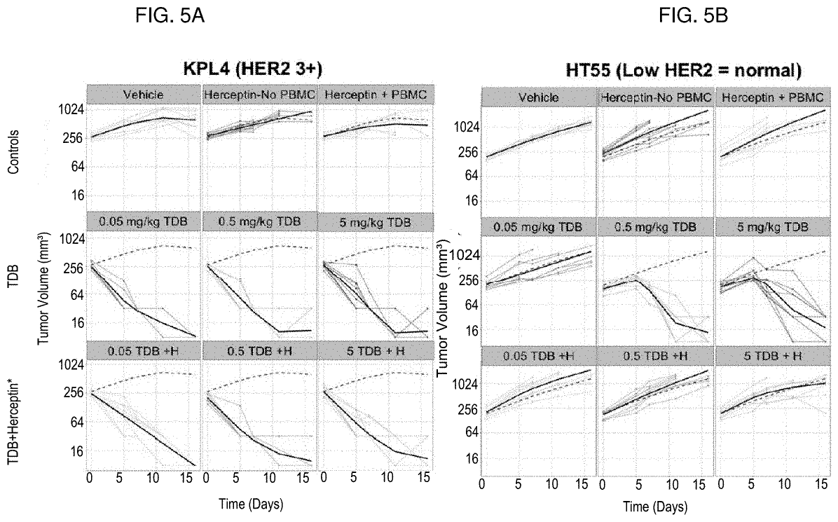

As shown in FIG. 5A, a single dose of BTRC4017A induced regression of KPL4 tumors at 0.05 mg/kg. A ten-fold greater dose of BTRC4017A was required to induce regression of HT55 tumors (FIG. 5B), indicating that the therapeutic index could be increased based on the high expression of HER2 in the HER2-positive tumors.

In co-treatment groups, 5.0 mg/kg trastuzumab-LALAPG was administered four hours before administration of BTRC4017A. Trastuzumab-LALAPG had no impact on BTRC4017A efficacy in targeting KPL4 tumors at any BTRC4017A dose level tested. In contrast, trastuzumab-LALAPG pretreatment abolished the BTRC4017A activity at all BTRC4017A dose-levels in the HT55 tumors. These data demonstrate that the impact of trastuzumab-LALAPG pretreatment on BTRC4017A activity is dramatically different between tumors that express different levels of HER2. In particular, activity was retained in KPL4 tumors (HER2-amplified), but abolished in HT55 tumors, which express HER2 at similar levels as normal human tissues.

These data suggest that treatment with trastuzumab prior to BTRC4017A administration can reduce the risk of on-target/off-tumor toxicity of BTRC4017A on normal tissues that express low levels of HER2, while maintaining antitumor activity in HER2 overexpressing tumors. Co-administration of trastuzumab with BTRC4017A in in vivo mouse efficacy studies did not impair the anti-tumor activity of BTRC4017A on HER2-positive tumors, but completely abolished BTRC4017A anti-tumor activity on low HER2-expressing tumors that were representative of HER2 expression levels on normal tissues. Without wishing to be bound by theory, trastuzumab may saturate HER2 (thereby preventing BTRC4017A binding) in low-expressing HER2 normal tissues, while being unable to saturate HER2 in tumors that express higher densities of HER2. Thus, co-administration of trastuzumab before each dose of BTRC4017A can mitigate off-tumor/on-target toxicities of BTRC4017A in HER2-expressing normal tissues while not significantly impacting anti-tumor activity of BTRC4017A in patients with HER2-positive tumors. In this way, co-administration of trastuzumab can increase the therapeutic index of BTRC4017A.

### Example 2. Fractionated, Dose-Escalation Dosing Regimens for Treatment of HER2-Positive Cancers with BTRC4017A and Trastuzumab

To mitigate potential cytokine-driven toxicities, BTRC4017A is administered in a fractionated dosing regimen in Cycle 1 (C1), wherein the first dose is less than a second dose. In a two-step fractionated dosing regimen, the second dose in C1 is less than a third dose. Cycle 2 and any necessary subsequent cycles involve a single administration of a BTRC4017A dose equivalent to the highest dose of BTRC4017A in C1.

Trastuzumab is administered on Day −1 of C1 in order to appropriately distinguish between any infusion related reactions (IRRs) that may be associated with BTRC4017A versus trastuzumab. All subsequent trastuzumab doses for Cycle 2 (C2) and onwards are administered on Day 1 of the cycle, prior to administration of BTRC4017A. A summary of trastuzumab administration procedures is provided in Table 4, below:

TABLE 4
Trastuzumab Infusion Times and Observation Periods

| | | | | C2 Day1 |
|---|---|---|---|---|
| | | C1 Day1 | | and beyond |
| | | Post- | | Post- |
| | | trastuzumab | | Trastuzumab |
| Timing of | | infusion | C2 Day1 and | infusion |
| Last | C1 Day-1 | observation | beyond | Observation |
| Trastuzumab | Trastuzumab | time prior to | Trastuzumab | Time |
| Dose prior to | Dose/Infusion | BTRC4017A | dose/infusion | prior to |
| C1 Day1 | Time | dosing | time | BTRC4017A |
| >4 weeks | 8 mg/kg over | 4-24 hours | 6 mg/kg over | 0.5-2 hours |
| | 90 minutes | | 30-90 minutes | |
| 3 weeks | 6 mg/kg over | 0.5-2 hours | 6 mg/kg over | 0.5-2 hours |
| (+7 days) | 30-60 minutes | | 30-90 minutes | |

C1 Day-1 = Cycle 1 Day-1;
C1 Day1 = Cycle 1 Day 1;
C2 Day1 = Cycle 2 Day 1

BTRC4017A is administered via fractionated dosing during the first 21-day cycle and on the first day of each subsequent cycle for up to 17 cycles. BTRC4017A administration is by flat (fixed) dosing, independent of body weight, by intravenous infusion using standard medical syringes and syringe pumps or intravenous bags where applicable. Drug product is delivered by syringe pump via an intravenous infusion set or intravenous bag with a final BTRC4017A volume determined by the dose. For the initial low doses, BTRC4017A will be only administered via a peripheral catheter. The specific BTRC4017A dose determines the appropriate dosing concentrations, volumes, infusion times, and also dictates the specific administration apparatus (e.g., peripheral catheter vs. syringe pump vs. IV bags) to be used.

Beginning on Day 1 of the C1, subjects receive escalating doses of BTRC4017A on Days 1 and 8 or on Days 1, 8, and 15 for a one-step and two-step fractionated regimen, respectively. In Cycle 2 and beyond, BTRC4017A is given as a single dose only on Day 1 of each 21-day cycle. The BTRC4017A may be given up to ±2 days from the scheduled date (i.e., with a minimum of 19 days between doses) for logistic/scheduling reasons.

Corticosteroid premedication consisting of dexamethasone (20 mg intravenous) or methylprednisolone (80 mg intravenous) is administered one hour prior to the administration of each BTRC4017A dose in Cycle 1. In addition, premedication with oral acetaminophen or paracetamol (e.g., 500-1000 mg) and/or 25-50 mg diphenhydramine is administered according to standard institutional practice prior to administration of BTRC4017A, unless contraindicated. Tocilizumab can be administered to patients intravenously at 8 mg/kg as necessary.

In the absence of a dose-limiting toxicity (DLT), unacceptable toxicity, or disease progression, patients deriving clinical benefit are offered continued treatment with BTRC4017A as a single agent or in combination with trastuzumab every 21 days up to a maximum of 17 cycles until progression or intolerable toxicity, whichever occurs first.

Disease status is assessed using the Response Evaluation Criteria in Solid Tumors (RECIST v1.1). Patients undergo tumor assessments at screening, during the study until treatment discontinuation, and at study termination. Immune-modified RECIST criteria are also used to characterize the patterns of responses associated with cancer immunotherapy. Immune-modified RECIST criteria can supplement standard RECIST v1.1 criteria to permit an integrated assessment of benefit and risk for patients. Pseudoprogression may be observed in the context of BTRC4017A and bispecific antibodies in general. As such, a patient who is deriving clinical benefit despite radiographic evidence of progressive disease as defined by standard RECIST v1.1 criteria may continue study treatment.

Adverse events are graded according to the National Cancer Institute Common Terminology Criteria for Adverse Events, Version 5.0 (NCI CTCAE v5.0), with the exception of cytokine release syndrome (CRS), which is graded according to the Modified Cytokine Release Syndrome Grading System (see Tables 1 and 2, above).

### OTHER EMBODIMENTS

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of treating or delaying the progression of a HER2-positive cancer in a subject in need thereof, the method comprising administering to the subject a treatment regimen comprising a HER2 antibody and a HER2 T cell-dependent bispecific antibody (TDB), the HER2 TDB comprising an anti-HER2 arm and an anti-CD3 arm, wherein the HER2 antibody and the HER2 TDB both bind domain IV of HER2, and wherein the treatment regimen results in an increased therapeutic index of the HER2 TDB as compared to treatment with the HER2 TDB in the absence of the HER2 antibody.

2. The method of claim 1, wherein the increased therapeutic index is associated with a decreased likelihood of experiencing an on-target/off-tumor effect as compared to treatment with the HER2 TDB in the absence of the HER2 antibody, and wherein the on-target/off-tumor effect is:
(a) a symptom of pulmonary toxicity selected from the group consisting of interstitial lung disease, acute respiratory distress syndrome, dyspnea, cough, fatigue, and pulmonary infiltrates;
(b) an elevated liver enzyme level;
(c) dry mouth;
(d) dry eyes;
(e) mucositis;
(f) esophagitis; or
(g) a urinary symptom.

3-5. (canceled)

6. The method of claim 1, wherein the increased therapeutic index is associated with a decreased likelihood of experiencing an immunogenic side effect as compared to treatment with the HER2 TDB in the absence of the HER2 antibody, and wherein the immunogenic side effect is selected from the group consisting of an elevated level of anti-drug antibodies, an infusion/administration-related reaction (ARR), cardiac dysfunction, a pulmonary reaction, and cytokine release syndrome.

7. (canceled)

8. The method of claim 1, wherein the HER2 TDB and the HER2 antibody bind competitively to domain IV of HER2.

9. The method of claim 1, wherein the HER2 antibody comprises:
(i) a complementarity-determining region (CDR)-H1 comprising the amino acid sequence of SEQ ID NO: 1;
(ii) a CDR-H2 comprising the amino acid sequence of SEQ ID NO: 2;
(iii) a CDR-H3 comprising the amino acid sequence of SEQ ID NO: 3;
(iv) a CDR-L1 comprising the amino acid sequence of SEQ ID NO: 4;
(v) a CDR-L2 comprising the amino acid sequence of SEQ ID NO: 5; and
(vi) a CDR-L3 comprising the amino acid sequence of SEQ ID NO: 6.

10. The method of claim 1, wherein the HER2 antibody comprises (a) a variable heavy chain domain (V_{H}) comprising at least 95% sequence identity to the amino acid sequence of SEQ ID NO: 7; (b) a variable light chain domain (V_{L}) comprising at least 95% sequence identity to the amino acid sequence of SEQ ID NO: 8; or (c) a V_{H} as in (a) and a V_{L} as in (b).

11. (canceled)

12. The method of claim 1, wherein the HER2 antibody is monospecific or a full-length antibody comprising an Fc region.

13. (canceled)

14. The method of claim 1, wherein the HER2 antibody is trastuzumab or an Fc-modified trastuzumab variant.

15. (canceled)

16. The method of claim 14, wherein the Fc-modified trastuzumab variant comprises one or more amino acid modifications that reduces effector function.

17-19. (canceled)

20. The method of claim 1, wherein the anti-HER2 arm of the HER2 TDB comprises a HER2 binding domain comprising:
(i) a CDR-H1 comprising the amino acid sequence of SEQ ID NO: 1;
(ii) a CDR-H2 comprising the amino acid sequence of SEQ ID NO: 2;
(iii) a CDR-H3 comprising the amino acid sequence of SEQ ID NO: 3;
(iv) a CDR-L1 comprising the amino acid sequence of SEQ ID NO: 4;
(v) a CDR-L2 comprising the amino acid sequence of SEQ ID NO: 5; and
(vi) a CDR-L3 comprising the amino acid sequence of SEQ ID NO: 6.

21. The method of claim 20, wherein the HER2 binding domain comprises (a) a V_{H} comprising at least 95% sequence identity to the amino acid sequence of SEQ ID NO: 7; (b) a V_{L} comprising at least 95% sequence identity to the amino acid sequence of SEQ ID NO: 8; or (c) a V_{H} as in (a) and a V_{L} as in (b).

22. (canceled)

23. The method of claim 1, wherein the anti-CD3 arm of the HER2 TDB comprises a CD3 binding domain comprising:
(i) a CDR-H1 comprising the amino acid sequence of SEQ ID NO: 9;
(ii) a CDR-H2 comprising the amino acid sequence of SEQ ID NO: 10;
(iii) a CDR-H3 comprising the amino acid sequence of SEQ ID NO: 11;
(iv) a CDR-L1 comprising the amino acid sequence of SEQ ID NO: 12;
(v) a CDR-L2 comprising the amino acid sequence of SEQ ID NO: 13; and
(vi) a CDR-L3 comprising the amino acid sequence of SEQ ID NO: 14.

24. The method of claim 23, wherein the CD3 binding domain comprises (a) a V_{H} comprising at least 95% sequence identity to the amino acid sequence of SEQ ID NO: 15; (b) a variable V_{L} comprising at least 95% sequence identity to the amino acid sequence of SEQ ID NO: 16; or (c) a V_{H} as in (a) and a V_{L} as in (b).

25. (canceled)

26. The method of claim 24, wherein (i) the anti-HER2 arm of the HER2 TDB comprises a HER2 binding domain comprising (a) a V_{H} comprising an amino acid sequence of SEQ ID NO: 7 and (b) a V_{L} comprising an amino acid sequence of SEQ ID NO: 8, and (ii) the anti-CD3 arm of the HER2 TDB comprises a CD3 binding domain comprising (a) a V_{H} comprising an amino acid sequence of SEQ ID NO: 15 and (b) a V_{L} comprising an amino acid sequence of SEQ ID NO: 16.

27. The method of claim 1, wherein the HER2 TDB is a full-length antibody comprising a modified Fc region.

28. The method of claim 27, wherein the modified Fc region comprises one or more substitution mutations that reduces effector function of the HER2 TDB.

29-30. (canceled)

31. The method of claim 28, wherein the one or more substitution mutations comprise an aglycosylation site mutation.

32-35. (canceled)

36. The method of claim 1, wherein the HER2 TDB comprises one or more heavy chain constant domains, wherein the one or more heavy chain constant domains are selected from a first CH1 (CH1₁) domain, a first CH2 (CH2₁) domain, a first CH3 (CH3₁) domain, a second CH1 (CH1₂) domain, second CH2 (CH2₂) domain, and a second CH3 (CH3₂) domain.

37. The method of claim 36, wherein at least one of the one or more heavy chain constant domains is paired with another heavy chain constant domain, wherein:
(i) the CH3₁ and CH3₂ domains each comprise a protuberance or cavity, and wherein the protuberance or cavity in the CH3₁ domain is positionable in the cavity or protuberance, respectively, in the CH3₂ domain; or
(ii) the CH2₁ and CH2₂ domains each comprise a protuberance or cavity, and wherein the protuberance or cavity in the CH2₁ domain is positionable in the cavity or protuberance, respectively, in the CH2₂ domain.

38. A method of treating or delaying the progression of a HER2-positive cancer in a subject in need thereof, the method comprising administering to the subject a treatment regimen comprising a HER2 antibody and a HER2 TDB, wherein (a) the HER2 antibody is trastuzumab or an Fc-modified trastuzumab variant, and (b) the HER2 TDB comprises an anti-HER2 arm and an anti-CD3 arm, wherein the anti-HER2 arm comprises a HER2 binding domain comprising:
(i) a CDR-H1 comprising the amino acid sequence of SEQ ID NO: 1;
(ii) a CDR-H2 comprising the amino acid sequence of SEQ ID NO: 2;
(iii) a CDR-H3 comprising the amino acid sequence of SEQ ID NO: 3;
(iv) a CDR-L1 comprising the amino acid sequence of SEQ ID NO: 4;
(v) a CDR-L2 comprising the amino acid sequence of SEQ ID NO: 5; and
(vi) a CDR-L3 comprising the amino acid sequence of SEQ ID NO: 6; and
wherein the anti-CD3 arm comprises a CD3 binding domain comprising:
(i) a CDR-H1 comprising the amino acid sequence of SEQ ID NO: 9;
(ii) a CDR-H2 comprising the amino acid sequence of SEQ ID NO: 10;
(iii) a CDR-H3 comprising the amino acid sequence of SEQ ID NO: 11;
(iv) a CDR-L1 comprising the amino acid sequence of SEQ ID NO: 12;
(v) a CDR-L2 comprising the amino acid sequence of SEQ ID NO: 13; and
(vi) a CDR-L3 comprising the amino acid sequence of SEQ ID NO: 14;
wherein the treatment regimen results in an increased therapeutic index of the HER2 TDB as compared to treatment with the HER2 TDB in the absence of the HER2 antibody.

39. The method of claim 1, wherein the HER2 antibody is administered prior to administration of the HER2 TDB.

40. The method of claim 1, wherein the HER2 antibody is administered at a dose of 5 mg/kg to 10 mg/kg or the HER2 TDB is administered at a fixed dose of 0.001 mg to 500 mg.

41. The method of claim 1, wherein the HER2 antibody is administered about once every three weeks or the HER2 TDB is administered about once every three weeks.

42-44. (canceled)

45. A method of treating or delaying the progression of a HER2-positive cancer in a subject in need thereof, the method comprising administering to the subject a treatment regimen comprising a HER2 antibody and a HER2 TDB, wherein the HER2 TDB comprises an anti-HER2 arm and an anti-CD3 arm, wherein the HER2 antibody and the HER2 TDB both bind domain IV of HER2, wherein the treatment regimen comprises:
(a) a first dose of the HER2 antibody;
(b) a first dosing cycle (C1) after the first dose of the HER2 antibody, the C1 comprising a first dose of the HER2 TDB (C1D1) and a second dose of the HER2 TDB (C1D2), wherein the C1D2 is greater than the C1D1;
(c) a second dosing cycle (C2) after the C1, the C2 comprising:
(i) a second dose of the HER2 antibody; and
(ii) an additional dose of the HER2 TDB (C2D1) after the second dose of the HER2 antibody, wherein the C2D1 is equivalent to the highest dose of the HER2 TDB of the C1.

46. The method of claim 45, wherein the first dose of the HER2 antibody is administered one day prior to the C1D1, and wherein the subject is monitored for a period of 30 minutes to 24 hours between the first dose of the HER2 antibody and the C1D1.

47. The method of claim 45, wherein the first dose of the HER2 antibody is from 5 mg/kg to 10 mg/kg or the second dose of the HER2 antibody is from 5 to 10 mg/kq.

48-50. (canceled)

51. The method of claim 45, wherein the first or second dose of the HER2 antibody is administered by infusion over a period of at least 30 minutes.

52. The method of claim 45, wherein the second dose of the HER2 antibody is administered on the same day as the C2D1.

53. The method of claim 45, wherein the C1D2 is at least two-fold the dose of the C1D1.

54. (canceled)

55. The method of claim 45, wherein the C1D1 is from 0.003 mg to 50 mg; the C1D2 is from 0.009 mg to 200 mg; or the C2D1 and the C1D2 are equivalent.

56-57. (canceled)

58. The method of claim 45, wherein the C1 further comprises a third dose of the HER2 TDB (C1D3), wherein the C1D3 is greater than the C1D2.

59. The method of claim 58, wherein the C1D1, the C1D2, and the C1D3 are cumulatively greater than a highest cleared dose of the HER2 TDB in a first dosing cycle of a one-step fractionation, dose-escalation dosing regimen.

60. (canceled)

61. The method of claim 58, wherein the C1D2 is from two-fold to ten-fold the dose of the C1D1; the C1D3 is from two-fold to three-fold the dose of the C1D2; or the C2D1 and the C1D3 are equivalent.

62-63. (canceled)

64. The method of claim 58, wherein the C1D1 is from 0.01 mg to 20 mg; the C1D2 is from 0.1 mg to 100 mg; or the C1D3 is from 1 mg to 200 mg.

65-66. (canceled)

67. The method of claim 58, wherein the method comprises administering to the subject the C1D1, the C1D2, and the C1D3 on or about Days 1, 8, and 15, respectively, of the C1 or the method comprises administering to the subject the C2D1 on Day 1 of the C2.

68. The method of claim 45, wherein the length of the C1 is 21 days or the length of the C2 is 21 days.

69. (canceled)

70. The method of claim 45, wherein the method comprises administering to the subject the C2D1 on Day 1 of the C2.

71. The method of claim 45, wherein the treatment regimen comprises one or more additional dosing cycles.

72. (canceled)

73. The method of claim 71, wherein the length of each of the one or more additional dosing cycles is 21 days.

74. The method of claim 71, wherein each of the one or more additional dosing cycles comprises a single dose of the HER2 antibody and a single dose of the HER2 TDB.

75. The method of claim 74, wherein the method comprises administering to the subject the HER2 antibody and the HER2 TDB on Day 1 of each of the one or more additional dosing cycles.

76. The method of claim 75, wherein the HER2 antibody is administered prior to the HER2 TDB on Day 1 of each of the one or more additional dosing cycles.

77. A method of treating or delaying the progression of a HER2-positive cancer in a subject in need thereof, the method comprising administering to the subject a treatment regimen comprising a HER2 TDB, wherein the treatment regimen comprises:
(a) a first cycle (C1) comprising a first dose of the HER2 TDB (C1D1) and a second dose of the HER2 TDB (C1D2), wherein the C1D2 is greater than the C1D1; and
(b) a second cycle (C2) comprising an additional dose of the HER2 TDB (C2D1), wherein the C2D1 is equivalent to the highest dose of the HER2 TDB of the C1.

78. The method of claim 77, wherein the C1D2 is at least two-fold the dose of the C1D1.

79. (canceled)

80. The method of claim 77, wherein the C1D1 is from 0.003 mg to about 10 mg; the C1D2 is from 0.009 to about 20 mg; or the C2D1 and the C1D2 are equivalent.

81-82. (canceled)

83. The method of claim 77, wherein the C1 further comprises a third dose of the HER2 TDB (C1D3) which is greater than the C1D2.

84. The method of claim 83, wherein the C1D1, the C1D2, and the C1D3 are cumulatively greater than a highest cleared dose of the HER2 TDB in a first dosing cycle of a one-step fractionation, dose-escalation dosing regimen.

85. (canceled)

86. The method of claim 83, wherein the C1D2 is from two-fold to ten-fold the dose of the C1D1; the C1D3 is from two-fold to three-fold the dose of the C1D2; or the C2D1 and the C1D3 are equivalent.

87-88. (canceled)

89. The method of claim 83, wherein the C1D1 is from 0.01 mg to 20 mg; the C1D2 is from 0.1 mg to 100 mg; or the C1D3 is from 1 mg to 200 mg.

90-91. (canceled)

92. The method of claim 83, wherein the method comprises administering to the subject the C1D1, the C1D2, and the C1D3 on or about Days 1, 8, and 15, respectively, of the C1.

93. The method of claim 77, wherein the length of the C1 is 21 days or the length of the C2 is 21 days.

94. (canceled)

95. The method of claim 77, wherein the method comprises administering to the subject the C2D1 on Day 1 of the C2.

96. The method of claim 77, wherein the treatment regimen comprises one or more additional dosing cycles.

97. (canceled)

98. The method of claim 96, wherein the length of each of the one or more additional dosing cycles is 21 days.

99. The method of claim 96, wherein each of the one or more additional dosing cycles comprises a single dose of the HER2 TDB.

100. The method of claim 96, wherein the method comprises administering to the subject the HER2 TDB on Day 1 of each of the one or more additional dosing cycles.

101. The method of claim 45, wherein the treatment regimen results in an increased therapeutic index of the HER2 TDB as compared to a control treatment regimen.

102. The method of claim 1, wherein the HER2 antibody or the HER2 TDB are administered by intravenous infusion.

103. The method of claim 1, further comprising administering one or more additional therapeutic agents.

104. The method of claim 103, wherein the one or more additional therapeutic agents are selected from the group consisting of tocilizumab, a corticosteroid, a PD-1 axis antagonist, and an antibody-drug conjugate.

105. The method of claim 104, wherein the PD-1 axis binding antagonist is selected from the group consisting of a PD-L1 binding antagonist, a PD-1 binding antagonist, and a PD-L2 binding antagonist.

106. The method of claim 105, wherein the PD-1 axis binding antagonist is:
(a) a PD-L1 binding antagonist selected from the group consisting of MPDL3280A (atezolizumab), MDX-1105, and MED14736;
(b) a PD-1 binding antagonist selected from the group consisting of MDX-1106 (nivolumab), MK-3475 (pembrolizumab), and AMP-224; or
(c) a PD-L2 binding antagonist, wherein the PD-L2 binding antagonist is an antibody or an immunoadhesin.

107-111. (canceled)

112. The method of claim 1, wherein the subject has been administered trastuzumab in a previous treatment regimen.

113. The method of claim 1, wherein the HER2-positive cancer is a HER2-positive solid tumor or a locally advanced or metastatic HER2-positive cancer.

114. (canceled)

115. The method of claim 1, wherein the HER2-positive cancer is a HER2-positive breast cancer, a HER2-positive gastric cancer, a HER2-positive gastroesophageal junction cancer, a HER2-positive colorectal cancer, a HER2-positive lung cancer, a HER2-positive pancreatic cancer, a HER2-positive bladder cancer, a HER2-positive salivary duct cancer, a HER2-positive ovarian cancer, or a HER2-positive endometrial cancer.

116-118. (canceled)
